(19) United States
(12) Patent Application Publication
(10) Pub. No.: US 2019/0000348 A1
(43) Pub. Date: Jan. 3, 2019

(54) WIRELESS COMMUNICATION SYSTEM, METHOD FOR WIRELESS COMMUNICATION, AND WIRELESS DEVICE

(71) Applicant: TOHOKU UNIVERSITY, Sendai-shi (JP)

(72) Inventors: Noriharu SUEMATSU, Sendai-shi (JP); Suguru KAMEDA, Sendai-shi (JP); Mizuki MOTOYOSHI, Sendai-shi (JP)

(73) Assignee: TOHOKU UNIVERSITY, Sendai-shi (JP)

(21) Appl. No.: 16/124,980

(22) Filed: Sep. 7, 2018

Related U.S. Application Data
(63) Continuation of application No. PCT/JP2016/057643, filed on Mar. 10, 2016.

(51) Int. Cl. A61B 5/07; A61B 90/98

(52) U.S. Cl. CPC A61B 5/073; H04W 4/80; A61B 90/98

(57) ABSTRACT

A wireless communication system includes a first wireless device configured to be introduced inside a living body and including an antenna; and a second wireless device configured to communicate with the first wireless device via the antenna. The wireless communication system carries out communication between the first wireless device and the second wireless device via the antenna at a first frequency when the first wireless device is outside the living body and carries out the communication at a second frequency lower than the first frequency when the first wireless device is inside the living body.

### CROSS-REFERENCE TO RELATED APPLICATION

This application is a continuation application of International Application PCT/JP2016/057643, filed on Mar. 10, 2016 and designated the U.S., the entire contents of which are incorporated herein by reference.

### FIELD

The embodiments discussed herein are related to a wireless communication system, a method for wireless communication, and a wireless device.

### BACKGROUND

A wireless system is known that includes a first wireless device and a second wireless device configured to communicate with the first wireless device via an antenna of the first wireless device, where the first wireless device can be introduced to the inside of a living body (for example, refer to Patent Document 1).

### LIST OF RELATED ART DOCUMENT

Patent Document 1: Japanese National Publication of International Patent Application No. 2014-525780

In such a wireless communication system, the communication between the first wireless device and the second wireless device is carried out when the first wireless device is not only inside a living body but also outside the living body.

The antenna of the first wireless device inside the living body is often in contact with biological fluids, for example, digestive fluid, inside the living body. In contrast, the antenna of the first wireless device outside the living body is often in contact with air. The permittivity of the biological fluid inside the living body differs from the permittivity of air.

Thus, the resonance frequency of the antenna of the first wireless device inside the living body often differs from the resonance frequency of the antenna of the first wireless device outside the living body. This may preclude communication between the first wireless device and the second wireless device regardless of whether the first wireless device is inside or outside the living body.

According to an aspect, a wireless communication system includes a first wireless device configured to be introduced inside a living body, the first wireless device comprising an antenna; and a second wireless device configured to communicate with the first wireless device via the antenna. The wireless communication system carries out communication between the first wireless device and the second wireless device via the antenna at a first frequency when the first wireless device is outside the living body and carries out the communication at a second frequency lower than the first frequency when the first wireless device is inside the living body.

According to another aspect, a method for wireless communication is carried out by a wireless communication system including a first wireless device including an antenna and configured to be introduced inside a living body and a second wireless device configured to communicate with the first wireless device via the antenna.

The method includes carrying out communication between the first wireless device and the second wireless device via the antenna at a first frequency when the first wireless device is outside the living body; and carrying out the communication at a second frequency lower than the first frequency when the first wireless device is inside the living body.

According to another aspect, a wireless device is configured to be introduced inside a living body and include an antenna. The wireless device includes a detector configured to detect whether the wireless device is inside the living body; a transmitter configured to transmit a signal via the antenna; and a controller configured to control the frequency of the signal to a first frequency when the wireless device is detected to be outside the living body, and control the frequency of the signal to a second frequency lower than the first frequency when the wireless device is detected to be inside the living body.

According to another aspect, a wireless device functions as a second wireless device configured to communicate with a first wireless device via an antenna of the first wireless device, the first wireless device being configured to be introduced inside a living body.

The wireless device includes a transmitter that transmits a signal; and a controller configured to control the frequency of the signal to a first frequency when the first wireless device is outside the living body, and control the frequency of the signal to a second frequency that is lower than the first frequency when the first wireless device is inside the living body.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating the configuration of a wireless communication system according to a first embodiment.

FIG. 2 is a block diagram illustrating the configuration of the reader in FIG. 1.

FIG. 3 is a block diagram illustrating the configuration of the tag device in FIG. 1.

FIG. 4 is a sequence diagram illustrating an example operation of the wireless communication system in FIG. 1.

FIG. 5 is a sequence diagram illustrating an example operation of the wireless communication system in FIG. 1.

FIG. 6 is a sequence diagram illustrating an example operation of a wireless communication system according to a modification to the first embodiment.

FIG. 7 is a block diagram illustrating the configuration of a reader according to the first modification to the first embodiment.

FIG. 8 is a block diagram illustrating the configuration of a tag device according to the first modification to the first embodiment.

FIG. 9 is a sequence diagram illustrating an example operation of a wireless communication system according to the first modification to the first embodiment.

FIG. 10 is a sequence diagram illustrating an example operation of the wireless communication system according to the first modification to the first embodiment.

FIG. 11 is a block diagram illustrating the configuration of a reader according to a second embodiment.

FIG. 12 is a block diagram illustrating the configuration of a tag device according to the second embodiment.

FIG. 13 is a sequence diagram illustrating an example operation of a wireless communication system according to the second embodiment.

FIG. 14 is a block diagram illustrating the configuration of a tag device according to a first modification to the second embodiment.

FIG. 15 is a sequence diagram illustrating an example operation of a wireless communication system according to the first modification to the second embodiment.

FIG. 16 is a sequence diagram illustrating an example operation of the wireless communication system according to the first modification to the second embodiment.

FIG. 17 is a block diagram illustrating the configuration of a reader according to a third embodiment.

FIG. 18 is a block diagram illustrating the configuration of a tag device according to the third embodiment.

FIG. 19 is a sequence diagram illustrating an example operation of a wireless communication system according to the third embodiment.

FIG. 20 is a block diagram illustrating the configuration of a tag device according to a first modification to the third embodiment.

FIG. 21 is a sequence diagram illustrating an example operation of a wireless communication system according to the first modification to the third embodiment.

FIG. 22 is a sequence diagram illustrating an example operation of the wireless communication system according the first modification to the third embodiment.

### DESCRIPTION OF EMBODIMENTS

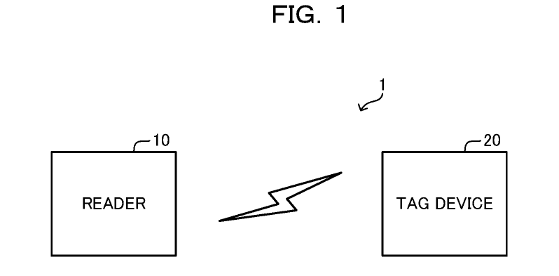

A wireless communication system, a method for wireless communication, and a wireless device according to embodiments of the present invention will now be described with reference to FIGS. 1 to 22.

### First Embodiment

(Configuration)

With reference to FIG. 1, a wireless communication system 1 according to a first embodiment includes a reader 10 and a tag device 20. In this embodiment, the wireless communication system 1 is a Radio Frequency IDentifier (RFID) system.

Besides the RFID system, the wireless communication system 1 may be any wireless communication system. For example, the wireless communication system 1 may carry out communication in accordance with a specific wireless communication scheme. Examples of the wireless communication scheme include Bluetooth Low Energy (BLE), ANT, ANT+, and ZigBee schemes. Bluetooth, ANT+, and ZigBee are trademarks. BLE may also be known as Bluetooth 4.0, Bluetooth Smart, or Bluetooth Smart Ready.

The tag device 20 may also be referred to as the RFID, RFID tag, wireless tag, or Integrated Circuit (IC) tag.

In this embodiment, the tag device 20 corresponds to a first wireless device. In this embodiment, the reader 10 corresponds to a second wireless device.

The tag device 20 can be introduced inside a living body (a human body, in this embodiment). In this embodiment, the tag device 20 can be introduced inside the living body through oral administration. For example, the tag device 20 may be attached to an artificial tooth. The tag device 20 may constitute at least a portion of the artificial tooth. In this embodiment, the artificial tooth is a partial denture. Alternatively, the artificial tooth may be a complete denture. Alternatively, the tag device 20 may be attached to a sensor that detects a physical quantity or otherwise constitute at least part of the sensor. Alternatively, the tag device 20 may be attached to a pharmaceutical formulation, such as a capsule or a tablet, or otherwise constitute at least a portion of the pharmaceutical formulation.

(Configuration of Reader)

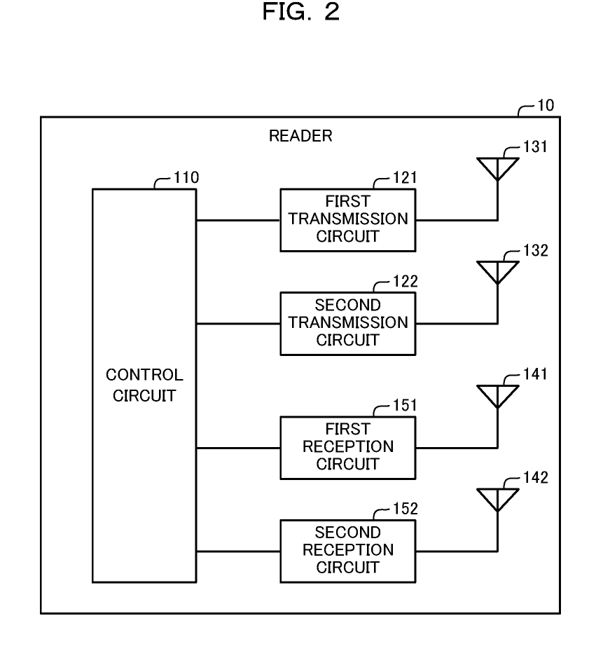

With reference to FIG. 2, the reader 10 includes a control circuit 110, a first transmission circuit 121, a second transmission circuit 122, a first transmission antenna 131, a second transmission antenna 132, a first reception antenna 141, a second reception antenna 142, a first reception circuit 151, and a second reception circuit 152.

In this embodiment, the first transmission circuit 121 and the second transmission circuit 122 correspond to transmitters. In this embodiment, the first reception circuit 151 and the second reception circuit 152 correspond to receivers. In this embodiment, the control circuit 110 corresponds to a controller.

In this embodiment, at least part of the reader 10 is composed of a Large Scale Integration (LSI) circuit. The at least part of the reader 10 may be composed of a programmable logic circuit (for example, a Programmable Logic Device (PLD) or a Field-Programmable Gate Array (FPGA)).

The reader 10 includes a processor and a storage unit. At least some of the functions of the reader 10 may be provided through execution of programs stored in the storage unit by the processor. For example, the processor may include a Central Processing Unit (CPU), a Micro-Processing Unit (MPU), or a Digital Signal Processor (DSP). The storage unit may include a Random Access Memory (RAM), a semiconductor memory, or an organic memory.

The reader 10 may constitute at least part of a mobile phone, a smart phone, or a personal computer, for example. The reader 10 may be in connection with a mobile phone, a smart phone, or a personal computer, for example.

The first transmission circuit 121 transmits a request signal having a carrier wave having a first frequency (in other words, first request signal) via the first transmission antenna 131. In this embodiment, the first frequency is included in a first frequency band among multiple frequency bands collectively referred to as an Industrial-Scientific-Medical (ISM) band. In this embodiment, the first frequency is in the 5.8 GHz band (from 5.725 to 5.875 GHz, for example). In this embodiment, the request signal requests the tag device 20 to transmit information. In this embodiment, the request signal includes a first signal component, a second signal component, and a third signal component, which are continuous along a time axis.

The first signal component and the third signal component are unmodulated waves (in other words, carrier waves). The second signal component is a modulated wave (in other words, radio wave obtained by modulating a carrier wave). For example, the second signal component represents identification information. The identification information identifies the information requested to be transmitted from the tag device 20.

In this embodiment, the first signal component, the second signal component, and the third signal component have predetermined durations of a first length, a second length, and a third length, respectively.

The second transmission circuit 122 transmits a request signal having a carrier wave having a second frequency (in other words, second request signal) via the second transmission antenna 132. The second frequency is lower than the first frequency. In this embodiment, the second frequency is lower than the half of the first frequency. In this embodiment, the second frequency is included in a second frequency band lower than the first frequency band among multiple frequency bands collectively referred to as an ISM band. In this embodiment, the second frequency is included in the 2.45 GHz band (from 2.4 to 2.5 GHz, for example).

The first reception circuit 151 receives a response signal having a carrier wave having the first frequency (in other words, first response signal) via the first reception antenna 141. In this embodiment, the response signal represents information specified by the identification information (in other words, response information). The response information includes at least one of the information stored in the tag device 20 and the information generated by the tag device 20, as described below. In this embodiment, the response signal is generated through modulation of at least a portion of the third signal component of the request signal as a result of being reflected at the tag device 20, as described below.

The second reception circuit 152 receives a response signal having a carrier wave having the second frequency (in other words, second response signal) via the second reception antenna 142.

The control circuit 110 controls the first transmission circuit 121 to start the transmission of the request signal. The control circuit 110 also controls the first transmission circuit 121 to end the transmission of the request signal. The control circuit 110 controls the second transmission circuit 122 in a similar manner to the first transmission circuit 121.

The control circuit 110 controls the first reception circuit 151 to start the stand-by for reception of the response signal. The control circuit 110 also controls the first reception circuit 151 to end the stand-by for reception of the response signal. The control circuit 110 controls the second reception circuit 152 in a similar manner to the first reception circuit 151.

In this embodiment, the control circuit 110 simultaneously instructs the first transmission circuit 121 and the second transmission circuit 122 to start transmission of request signals and simultaneously instructs the first reception circuit 151 and the second reception circuit 152 to start the stand-by for reception of the response signals. In this embodiment, the control circuit 110 controls the first reception circuit 151 and the second reception circuit 152 such that the stand-by for reception of the response signals starts substantially simultaneously with the start of the transmission of the request signals. The stand-by for the reception of the response signals may start after a predetermined delay time from the start of the transmission of the request signals.

The control circuit 110 may control the first transmission circuit 121 and the second transmission circuit 122 so as to alternately carry out the transmission of the request signal by the first transmission circuit 121 and the transmission of the request signal by the second transmission circuit 122.

In such a case, the control circuit 110 controls the first reception circuit 151 such that the first reception circuit 151 enters the stand-by for reception of the response signal for at least a portion of the period during which the request signal is transmitted by the first transmission circuit 121. Also in such a case, the control circuit 110 controls the second reception circuit 152 such that the second reception circuit 152 enters the stand-by for reception of the response signal for at least a portion of the period during which the request signal is transmitted by the second transmission circuit 122.

In this embodiment, the control circuit 110 obtains the response information on the basis of only the signal having the larger intensity among the first response signal received by the first reception circuit 151 and the second response signal received by the second reception circuit 152. For example, the intensity of the signal is a parameter that increases as the electrical energy and/or the amplitude of the signal increases.

The control circuit 110 may detect that the tag device 20 has been introduced from the outside to the inside of a living body on the basis of the intensity of the first response signal received by the first reception circuit 151 and the intensity of the second response signal received by the second reception circuit 152. In such a case, the control circuit 110 may detect that the tag device 20 has been introduced from the outside to the inside of the living body through a variation in the intensity of the first response signal from being larger than the intensity of the second response signal to being smaller than the intensity of the second response signal.

The control circuit 110 may detect that the tag device 20 has been moved from the inside to the outside of the living body on the basis of the intensity of the first response signal received by the first reception circuit 151 and the intensity of the second response signal received by the second reception circuit 152. In such a case, the control circuit 110 may detect that the tag device 20 has been moved from the inside to the outside of the living body through a variation in the intensity of the second response signal from being larger than the intensity of the first response signal to being smaller than the intensity of the first response signal.

(Configuration of Tag Device)

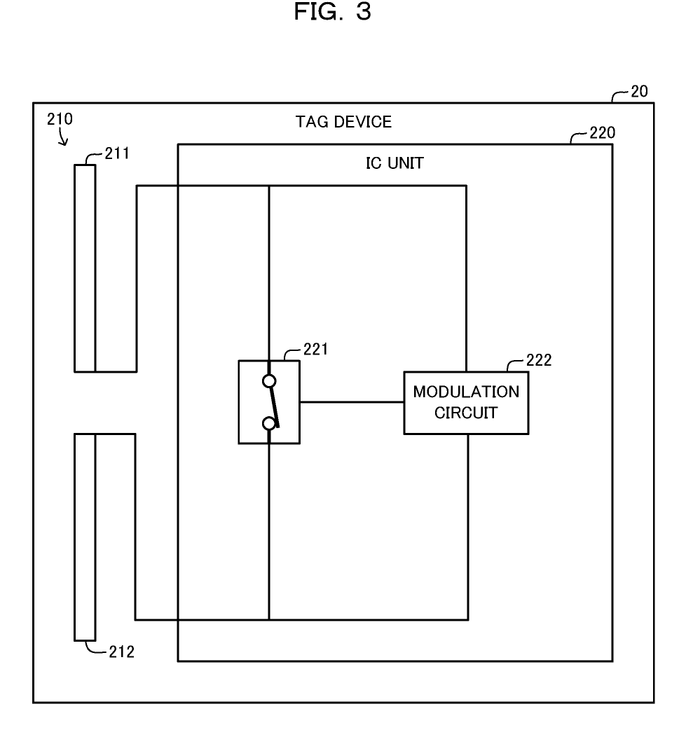

With reference to FIG. 3, the tag device 20 includes an antenna 210 and an IC unit 220. In this embodiment, the IC unit 220 corresponds to a transmitter.

In this embodiment, at least part of the tag device 20 is composed of an LSI circuit. The at least part of the tag device 20 may be composed of a programmable logic circuit. The tag device 20 includes a processor and a storage unit. At least some of the functions of the tag device 20 may be provided through execution of programs stored in the storage unit by the processor.

The antenna 210 includes a first antenna component 211 and a second antenna component 212. In this embodiment, the first antenna component 211 and the second antenna component 212 constitute a dipole antenna. In this embodiment, the first antenna component 211 and the second antenna component 212 have a meandering shape. The first antenna component 211 and the second antenna component 212 may have any shape (for example, a linear shape) besides a meandering shape.

In this embodiment, the resonance frequency of the antenna 210 is substantially identical to the first frequency during contact of the antenna 210 with air. In this embodiment, the resonance frequency of the antenna 210 is substantially identical to the second frequency during contact of the antenna 210 and a fluid inside the living body (for example, a digestive fluid, such as saliva, in this embodiment).

The antenna 210 may be of any type (such as a loop antenna, a plate antenna, or a flat antenna) besides a dipole antenna.

The IC unit 220 is in connection with the first antenna component 211 and the second antenna component 212. The IC unit 220 includes a switching device 221 and a modulation circuit 222.

In this embodiment, the tag device 20 is of a passive type. The IC unit 220 is activated by a potential difference generated between the first antenna component 211 and the second antenna component 212 when the antenna 210 receives signals.

In this embodiment, the IC unit 220 includes a rectifier and a capacitor (not illustrated). The current generated between the first antenna component 211 and the second antenna component 212 is rectified at the rectifier and stored in the capacitor for at least a portion of the period during which the first signal component of the request signal is received.

The IC unit 220 demodulates the second signal component for at least a portion of the period during which the second signal component of the request signal is received using the electrical energy stored in the capacitor. The IC unit 220 obtains identification information on the basis of the demodulated signal.

The switching device 221 switches between a short-circuited state in which the first antenna component 211 and the second antenna component 212 are short-circuited (in other words, connected) and a disconnected state in which the first antenna component 211 and the second antenna component 212 are cutoff (in other words, disconnected).

In this embodiment, the reflection intensity in a short-circuited state of the switching device 221 is larger than the reflection intensity in the cutoff state of the switching device 221. The reflection intensity refers to the intensity of a signal reflected at the tag device 20 among the signals received by the antenna 210 (in other words, signal transmitted by the tag device 20).

In other words, the electrical energy absorbed by the IC unit 220 from a signal received by the antenna 210 in the disconnected state of the switching device 221 is larger than that in the short-circuited state of the switching device 221.

The modulation circuit 222 controls the state of the switching device 221 on the basis of the information specified by the identification information (in other words, response information) using the electrical energy stored in the capacitor for at least a portion of the period during which the third signal component of the request signal is received. In this embodiment, the modulation circuit 222 controls the state of the switching device 221 such that the state of the switching device 221 corresponds to at least one bit representing response information.

In this embodiment, the short-circuited state corresponds to a bit value “1,” whereas the cutoff state corresponds to a bit value “0.” Alternatively, the short-circuited state may correspond to a bit value “0,” whereas the cutoff state may correspond to a bit value “1.”

In this embodiment, the response information contains information stored in the IC unit 220 in advance. For example, the response information may contain an identifier for identifying the tag device 20. If the tag device 20 includes a sensor for detecting a physical quantity, the response information may contain information representing the physical quantity detected by the sensor in addition to the information stored in the IC unit 220 in advance or instead of the information stored in the IC unit 220 in advance. The physical quantity is temperature, humidity, illuminance, pH, acceleration, angular velocity, pressure, or concentration of a subject, for example. Examples of the subject include a digestive fluid (e.g., saliva, gastric juice, intestinal juice, or pancreatic juice), blood, normal microbial flora, and infectious material (for example, bacteria or viruses).

In other words, in this embodiment, the IC unit 220 modulates the request signal received by the antenna 210 in accordance with an Amplitude Modulation (AM) scheme and transmits the modulated request signal as a response signal. In other words, in this embodiment, the tag device 20 transmits a response signal representing response information in accordance with a backscattering scheme.

The IC unit 220 may perform modulation in accordance with a modulation scheme other than the AM scheme. For example, the modulation scheme may be a Frequency Modulation (FM) or a Phase Modulation (PM) scheme. Alternatively, the modulation scheme may be a combination of at least two of the AM, FM, and PM schemes, for example.

(Operation)

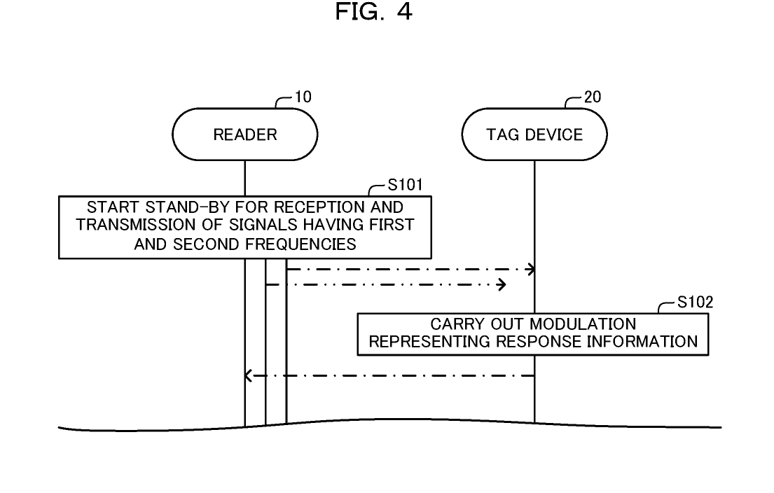

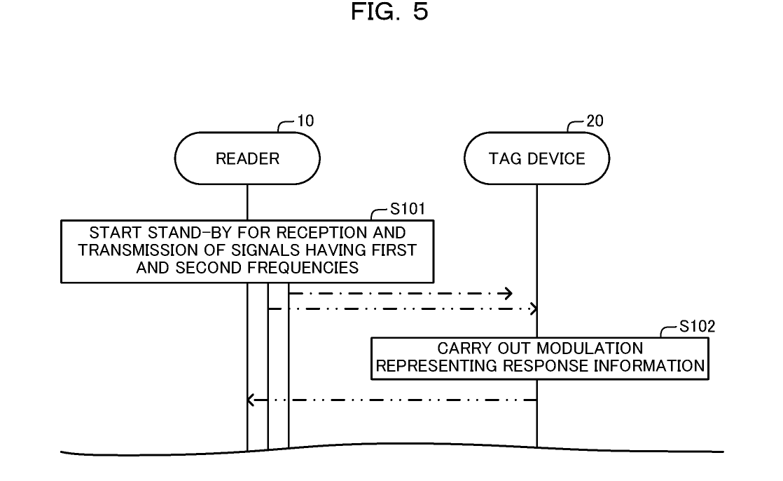

The operation of the wireless communication system 1 according to the first embodiment will now be explained with reference to FIGS. 4 and 5.

A case of the tag device 20 outside the living body will now be explained. In this case, the antenna 210 of the tag device 20 is in contact with air.

The reader 10 starts transmission of a request signal having a carrier wave having the first frequency (in other words, first request signal) and a request signal having a carrier wave having the second frequency (in other words, second request signal) and starts the stand-by for reception of a response signal having a carrier wave having the first frequency (in other words, first response signal) and a response signal having a carrier wave having the second frequency (in other words, second response signal) (Step S101 in FIG. 4).

The tag device 20 receives the first request signal and the second request signal. Since the antenna 210 is in contact with air, the resonance frequency of the antenna 210 is substantially identical to the first frequency. Thus, the intensity of the first request signal received by the antenna 210 is larger than the intensity of the second request signal received by the antenna 210.

The tag device 20 rectifies the current generated between the first antenna component 211 and the second antenna component 212 at the rectifier and stores a charge in the capacitor for at least a portion of the period during which the first signal component of the request signal is received.

The tag device 20 demodulates the second signal component of the request signal for at least a portion of the period during which the second signal component is received, and obtains identification information on the basis of the demodulated signal, using the electrical energy stored in the capacitor.

For at least a portion of the period during which the third signal component of the request signal is received, the tag device 20 modulates the request signal received by the antenna 210 on the basis of the response information and transmits the modulated request signal as a response signal, using the electrical energy stored in the capacitor (S102 in FIG. 4). In this embodiment, the tag device 20 transmits a response signal representing response information in accordance with a backscattering scheme.

In this embodiment, the response signal transmitted by the antenna 210 contains a first response signal component having a carrier wave having the first frequency (in other words, first component) and a second response signal component having a carrier wave having the second frequency (in other words, second component). Since the resonance frequency of the antenna 210 is substantially identical to the first frequency, the intensity of the first response signal component is larger than the intensity of the second response signal component.

The reader 10 receives the response signal transmitted by the tag device 20. The reader 10 obtains the response information on the basis of only the response signal component having the larger intensity among the first response signal component and the second response signal component of the received response signal. Thus, the reader 10 obtains the response information on the basis of only the first response signal component among the first response signal component and the second response signal component of the received response signal.

A case of the tag device 20 inside the living body will now be described. In this case, the antenna 210 of the tag device 20 is in contact with a fluid inside the living body (saliva, in this embodiment).

Similar to the operation explained above, the reader 10 starts transmission of the first request signal and transmission of the second request signal and starts the stand-by for reception of the first request signal and the stand-by for reception of the second request signal (S101 in FIG. 5).

The tag device 20 receives the first request signal and the second request signal. Since the antenna 210 is in contact with the fluid inside the living body, the resonance frequency of the antenna 210 is substantially identical to the second frequency. Thus, the intensity of the second request signal received by the antenna 210 is larger than the intensity of the first request signal received by the antenna 210.

The tag device 20 rectifies the current generated between the first antenna component 211 and the second antenna component 212 at the rectifier and stores a charge in the capacitor for at least a portion of the period during which the first signal component of the request signal is received.

The tag device 20 demodulates the second signal component for at least a portion of the period during which the second signal component of the request signal is received, and obtains the identification information on the basis of the demodulated signal, using the electrical energy stored in the capacitor.

For at least a portion of the period during which the third signal component of the request signal is received, the tag device 20 modulates the request signal received by the antenna 210 on the basis of the response information and transmits the modulated request signal as a response signal, using the electrical energy stored in the capacitor (Step S102 in FIG. 5). In this embodiment, the tag device 20 transmits a response signal representing response information in accordance with a backscattering scheme.

In this embodiment, the response signal transmitted by the antenna 210 contains a first response signal component having a carrier wave having the first frequency (in other words, first component) and the second response signal component having a carrier wave having the second frequency (in other words, second component). Since the resonance frequency of the antenna 210 is substantially identical to the second frequency, the intensity of the second response signal component is larger than the intensity of the first response signal component.

The reader 10 receives the response signal transmitted by the tag device 20. The reader 10 obtains the response information on the basis of only the response signal component having the larger intensity among the first response signal component and the second response signal component of the received response signal. Thus, the reader 10 obtains the response information on the basis of only the second response signal component among the first response signal component and the second response signal component of the received response signal.

As described above, the wireless communication system 1 according to the first embodiment carries out communication between the tag device 20 and the reader 10 via the antenna 210 at the first frequency when the tag device 20 is outside the living body. The wireless communication system 1 carries out communication between the tag device 20 and the reader 10 via the antenna 210 at the second frequency, which is lower than the first frequency, when the tag device 20 is inside the living body.

Thus, the reader 10 can communicate with the tag device 20 via the antenna 210 of the tag device 20 regardless of whether the tag device 20 is inside or outside the living body.

The reader 10 according to the first embodiment receives a signal having the first frequency and a signal having the second frequency.

Thus, the reader 10 can receive a signal having the first frequency when the tag device 20 is outside the living body. The reader 10 can receive a signal having the second frequency when the tag device 20 is inside the living body. Thus, the reader 10 can communicate with the tag device 20 via the antenna 210 of the tag device 20 regardless of whether the tag device 20 is inside or outside the living body.

The reader 10 according to the first embodiment transmits a signal having the first frequency and a signal having the second frequency.

Thus, the tag device 20 can receive the signal having the first frequency when the tag device 20 is outside the living body. The tag device 20 can receive the signal having the second frequency when the tag device 20 is inside the living body. Thus, the tag device 20 can communicate with the reader 10 via the antenna 210 of the tag device 20 regardless of whether the tag device 20 is inside or outside the living body.

The tag device 20 according to the first embodiment modulates the signal transmitted by the reader 10 and transmits the modulated signal.

Thus, the reader 10 can receive the signal having the first frequency when the tag device 20 is outside the living body. The reader 10 can receive the signal having the second frequency when the tag device 20 is inside the living body. Thus, the reader 10 can communicate with the tag device 20 via the antenna 210 of the tag device 20 regardless of whether the tag device 20 is inside or outside the living body.

The wireless communication system 1 may use a frequency included in the 2.45 GHz band (from 2.4 to 2.5 GHz, for example) as the first frequency and use a frequency included in the 900 MHz band (from 915 to 955 MHz, for example) as the second frequency.

Alternatively, the wireless communication system 1 may use a frequency included in the 5.8 GHz band (from 5.725 to 5.875 GHz, for example) as the first frequency and use a frequency included in the 900 MHz band (from 915 to 955 MHz, for example) as the second frequency.

Alternatively, the wireless communication system 1 may use a frequency included in the 60 GHz band (from 57 to 66 GHz, for example) as the first frequency and use a frequency included in the 5.8 GHz band (from 5.725 to 5.875 GHz, for example) as the second frequency.

Alternatively, the wireless communication system 1 may use a frequency included in the 24 GHz band (from 24 to 24.25 GHz, for example) as the first frequency and use a frequency included in the 5.8 GHz band (from 5.725 to 5.875 GHz, for example) as the second frequency.

The tag device 20 may include a modifier that modifies the resonance frequency of the antenna 210. For example, the modifier may include a first antenna extension, a second antenna extension, a first switching device, and a second switching device.

The first antenna extension is in connection with the first antenna component 211 via the first switching device. The first switching device switches between a state in which the first antenna component 211 and the first antenna extension are connected and a state in which the first antenna component 211 and the first antenna extension are disconnected.

The second antenna extension is in connection with the second antenna component 212 via the second switching device. The second switching device switches between a state in which the second antenna component 212 and the second antenna extension are connected and a state in which the second antenna component 212 and the second antenna extension are disconnected.

This can modify the ratio of the first frequency to the second frequency to a value different from the ratio of the permittivity of air to the permittivity of a fluid inside the living body. Thus, the frequency used for communication between the reader 10 and the tag device 20 can be selected from a wider range.

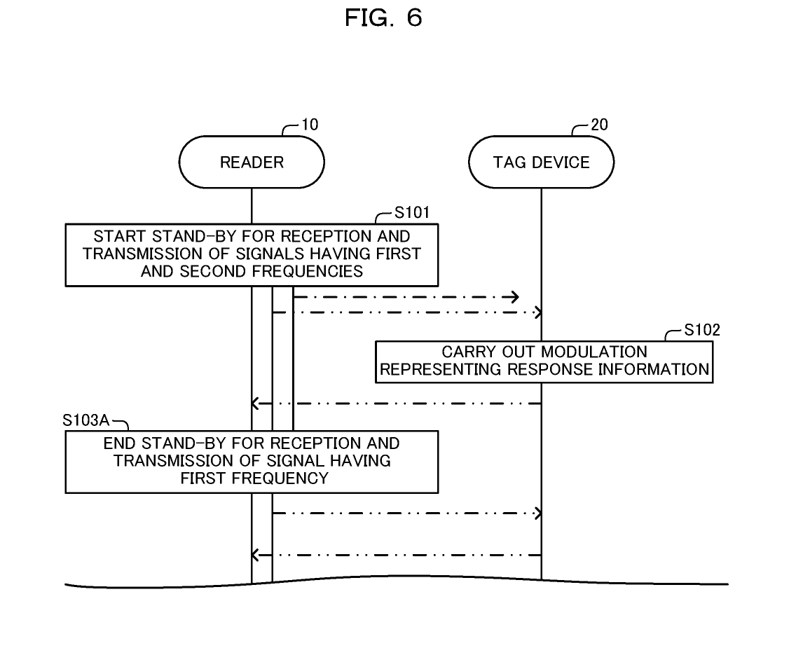

With reference to FIG. 6, the reader 10 may end the transmission of the first request signal and the stand-by for reception of the first response signal component of the response signal if the reader 10 receives the response signal and if the intensity of the first response signal component of the response signal is smaller than the intensity of the second response signal component of the response signal (S103A in FIG. 6). The reader 10 may end the transmission of the second request signal and the stand-by for reception of the second response signal component of the response signal if the reader 10 receives the response signal and if the intensity of the second response signal component of the received response signal is smaller than the intensity of the first response signal component of the received response signal.

This can reduce the electrical power consumption at the reader 10 during transmission of a request signal and the stand-by for reception of a response signal by the reader 10.

<First Modification to First Embodiment>

A wireless communication system according to a first modification to the first embodiment will now be described. The wireless communication system according to the first modification to the first embodiment differs from the wireless communication system according to the first embodiment in that the tag device detects whether it is inside a living body, the detected result is sent to the reader, and the reader controls the frequency to be used for communication on the basis of the detected result. The wireless communication system will now be described with focus on these differences. In the first modification to the first embodiment, components that are identical or very similar to those according to the first embodiment are indicated by the same reference signs.

(Configuration of Reader)

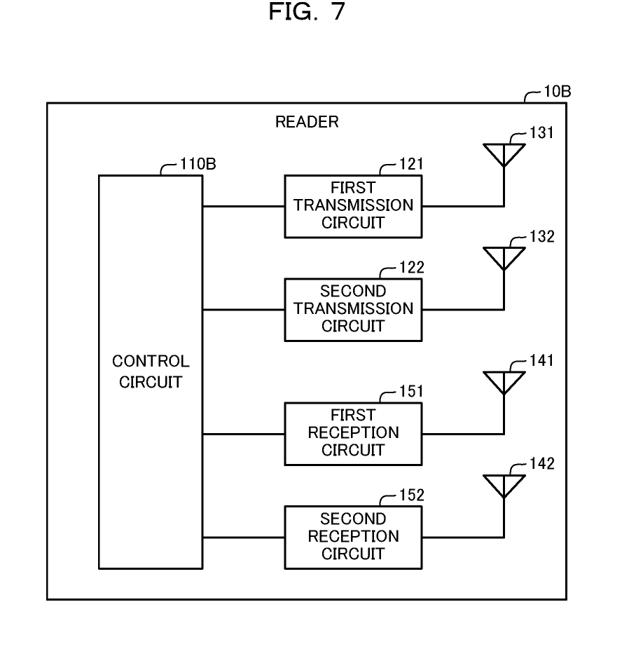

With reference to FIG. 7, a reader 10B according to the first modification to the first embodiment includes a control circuit 110B, a first transmission circuit 121, a second transmission circuit 122, a first transmission antenna 131, a second transmission antenna 132, a first reception antenna 141, a second reception antenna 142, a first reception circuit 151, and a second reception circuit 152.

In this modification, the first transmission circuit 121 and the second transmission circuit 122 correspond to transmitters. In this modification, the first reception circuit 151 and the second reception circuit 152 correspond to receivers. In this modification, the control circuit 110B corresponds to a controller.

The first transmission circuit 121 and the second transmission circuit 122 have the same configurations as those of the first transmission circuit 121 and the second transmission circuit 122 according to the first embodiment.

The first reception circuit 151 and the second reception circuit 152 operate in a similar manner as the first reception circuit 151 and the second reception circuit 152 according to the first embodiment and receive detection signals representing detection information. The detection information represents results detected by a sensor 223B of the tag device 20B, as described below.

The first reception circuit 151 receives a detection signal having a carrier wave having a first frequency (in other words, first detection signal) via the first reception antenna 141.

The second reception circuit 152 receives a detection signal having a carrier wave having a second frequency (in other words, second detection signal) via the second reception antenna 142.

The control circuit 110B controls the first transmission circuit 121 to start transmission of a request signal. The control circuit 110B controls the first transmission circuit 121 to end the transmission of the request signal. The control circuit 110B controls the second transmission circuit 122 in a similar manner to the first transmission circuit 121.

The control circuit 110B controls the first reception circuit 151 to start the stand-by for reception of a detection signal and a response signal. The control circuit 110B controls the first reception circuit 151 to end the stand-by for reception of the detection signal and the response signal. The control circuit 110B controls the second reception circuit 152 in a similar manner to the first reception circuit 151.

In this embodiment, the control circuit 110B simultaneously instructs the first transmission circuit 121 and the second transmission circuit 122 to start the transmission of request signals and simultaneously instructs the first reception circuit 151 and the second reception circuit 152 to start the stand-by for reception of detection signals and response signals. In this embodiment, the control circuit 110B controls the first reception circuit 151 and the second reception circuit 152 such that the stand-by for reception of the detection signals and the response signals starts substantially simultaneously with the start of the transmission of the request signals. The stand-by for reception of the detection signals and the response signals may start after a predetermined delay time from the start of the transmission of the request signals.

The control circuit 110B may control the first transmission circuit 121 and the second transmission circuit 122 so as to alternately carry out the transmission of the request signal by the first transmission circuit 121 and the transmission of the request signal by the second transmission circuit 122.

In such a case, the control circuit 110B controls the first reception circuit 151 such that the first reception circuit 151 enters the stand-by for reception of the detection signal and the response signal for at least a portion of the period during which the request signal is transmitted by the first transmission circuit 121. Also in such a case, the control circuit 110B controls the second reception circuit 152 such that the second reception circuit 152 enters the stand-by for reception of the detection signal and the response signal for at least a portion of the period during which the request signal is transmitted by the second transmission circuit 122.

The control circuit 110B obtains detection information on the basis of the detection signal having the larger intensity among the detection signal having a carrier wave having the first frequency (in other words, first detection signal) received by the first reception circuit 151 and the detection signal having a carrier wave having the second frequency (in other words, second detection signal) received by the second reception circuit 152. The control circuit 110B may obtain the detection information on the basis of both the first detection signal received by the first reception circuit 151 and the second detection signal received by the second reception circuit 152.

The control circuit 110B ends either the stand-by for reception and the transmission of the signal having a carrier wave having the first frequency or the stand-by for reception and the transmission of the signal having a carrier wave having the second frequency, on the basis of the detected result indicated by the obtained detection information.

In this modification, the control circuit 110B ends the transmission of the second request signal and the stand-by for reception of the response signal having a carrier wave having the second frequency (in other words, second response signal) and the second detection signal when the obtained detection information indicates that the tag device 20B is outside the living body. In this modification, the control circuit 110B ends the transmission of the first request signal and the stand-by for reception of a response signal having a carrier wave having the first frequency (in other words, first response signal) and the first detection signal when the obtained detection information indicates that the tag device 20B is inside the living body.

The control circuit 110B obtains response information on the basis of the response signal received by the first reception circuit 151 or the second reception circuit 152.

(Configuration of Tag Device)

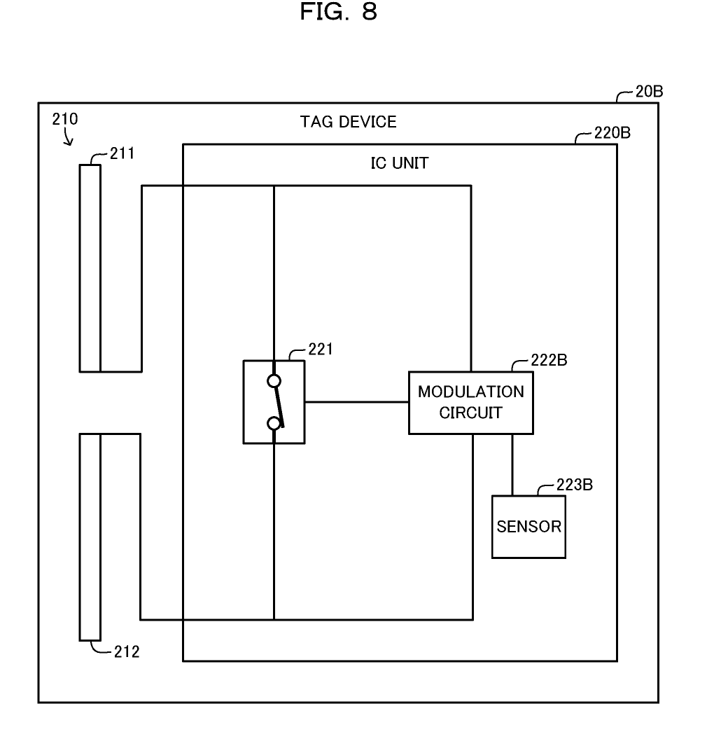

With reference to FIG. 8, the tag device 20B according to the first modification to the first embodiment includes an IC unit 220B in place of the IC unit 220 of the tag device 20 according to the first embodiment. The IC unit 220B includes a switching device 221, a modulation circuit 222B, and a sensor 223B. In this modification, the sensor 223B corresponds to a detector. In this modification, the IC unit 220B corresponds to a notifier.

The switching device 221 has the same configuration as that of the switching device 221 according to the first embodiment.

The sensor 223B detects whether the tag device 20B is inside a living body. In this modification, the sensor 223B detects temperature. The sensor 223B detects that the tag device 20B is inside the living body when the detected temperature is higher than or equal to a predetermined threshold (307K, for example) and detects that the tag device 20B is outside the living body when the detected temperature is lower than the threshold.

In addition to or instead of detecting temperature, the sensor 223B may detect whether the tag device 20B is inside the living body on the basis of a physical quality other than temperature. The physical quantity may be illuminance, pH, or concentration of a subject, for example. Examples of the subject include a digestive fluid (e.g., saliva, gastric juice, intestinal juice, or pancreatic juice), blood, and normal microbial flora.

In this modification, the sensor 223B operates by the electrical energy stored in the capacitor. The sensor 223B may include a battery, which provides electrical energy to the sensor 223B for operation.

The modulation circuit 222B operates in the same manner as the modulation circuit 222 for a period during which the first signal component and the second signal component of the request signal are received and operates in a different manner as the modulation circuit 222 for a period during which the third signal component of the request signal is received.

The modulation circuit 222B controls the state of the switching device 221 on the basis of information indicating the result detected by the sensor 223B (in other words, detection information) during a first sub-period, which is a portion of the period during which the third signal component of the request signal is received, using the electrical energy stored in the capacitor. In this modification, the modulation circuit 222B controls the state of the switching device 221 such that the state of the switching device 221 corresponds to at least one bit representing detection information.

In other words, in this modification, the tag device 20B transmits a detection signal representing detection information in accordance with a backscattering scheme. In this modification, the transmission of a detection signal corresponds to notification to the reader 10B of the result detected by the sensor 223B.

The IC unit 220B may perform modulation on the basis of the detection information in accordance with a modulation scheme other than the AM scheme.

The modulation circuit 222B controls the state of the switching device 221 on the basis of the response information for a second sub-period following the first sub-period in the period during which the third signal component of the request signal is received, using the electrical energy stored in the capacitor. In this embodiment, the modulation circuit 222B controls the state of the switching device 221 such that the state of the switching device 221 corresponds to at least one bit representing the response information.

In other words, in this modification, the tag device 20B transmits a response signal representing response information in accordance with a backscattering scheme. The IC unit 220B may perform modulation on the basis of the response information in accordance with a modulation scheme other than the AM scheme.

(Operation)

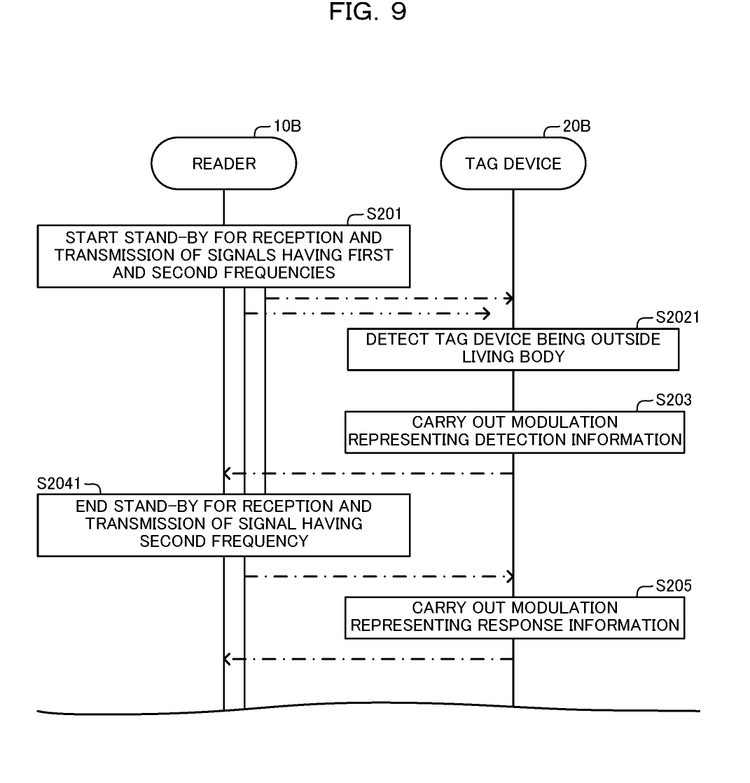

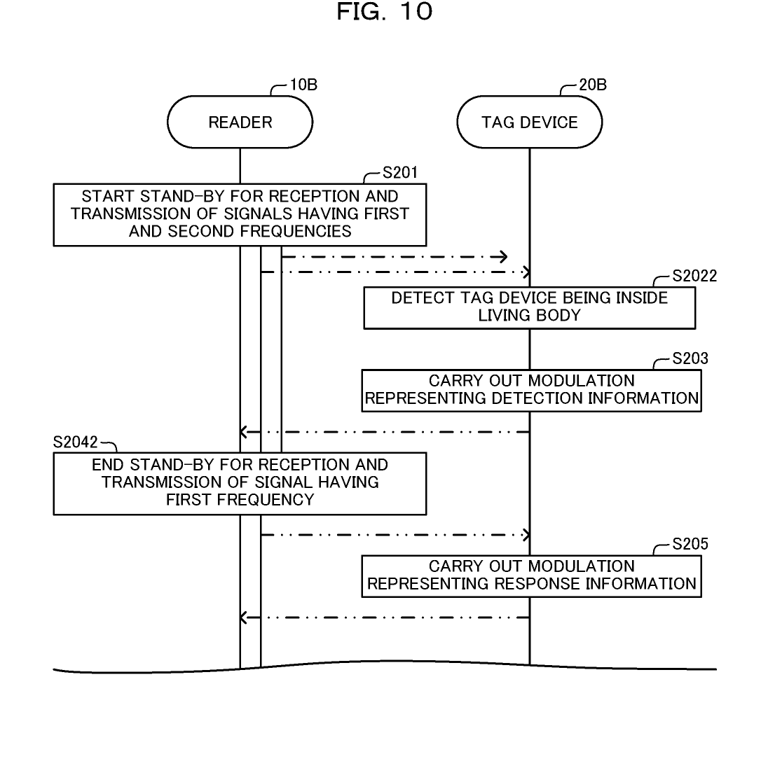

The operation of the wireless communication system 1 according to the first modification to the first embodiment will now be explained with reference to FIGS. 9 and 10.

A case of the tag device 20B outside the living body will now be explained. In this case, the antenna 210 of the tag device 20B is in contact with air.

The reader 10B starts the transmission of a request signal having a carrier wave having the first frequency (in other words, first request signal) and a request signal having a carrier wave having the second frequency (in other words, second request signal) and starts the stand-by for reception of signals having carrier waves having the first frequency (in other words, first detection signal and first response signal) and signals having carrier waves having the second frequency (in other words, second detection signal and second response signal) (Step S201 in FIG. 9).

The tag device 20B receives the first request signal and the second request signal. Since the antenna 210 is in contact with air, the resonance frequency of the antenna 210 is substantially identical to the first frequency. Thus, the intensity of the first request signal received by the antenna 210 is larger than the intensity of the second request signal received by the antenna 210.

The tag device 20B rectifies the current generated between the first antenna component 211 and the second antenna component 212 at the rectifier and stores a charge in the capacitor for at least a portion of the period during which the first signal component of the request signal is received.

The tag device 20B demodulates the second signal component of the request signal for at least a portion of the period during which the second signal component is received, and obtains identification information on the basis of the demodulated signal, using the electrical energy stored in the capacitor.

The tag device 20B detects whether the tag device 20B is inside the living body with the sensor 223B. In this case, the tag device 20B detects that the tag device 20B is not inside the living body (in other words, being outside the living body) (Step S2021 in FIG. 9).

For the first sub-period of the period during which the third signal component of the request signal is received, the tag device 20B modulates the request signal received by the antenna 210 using the electrical energy stored in the capacitor, on the basis of the detection information and transmits the modulated request signal as a detection signal (Step S203 in FIG. 9). In this modification, the tag device 20B transmits a detection signal representing detection information in accordance with a backscattering scheme.

In this modification, the detection signal transmitted by the antenna 210 includes a first detection signal component having a carrier wave having the first frequency (in other words, first component) and a second detection signal component having a carrier wave having the second frequency (in other words, second component). Since the resonance frequency of the antenna 210 is substantially identical to the first frequency, the intensity of the first detection signal component is larger than the intensity of the second detection signal component.

The reader 10B receives the detection signal transmitted by the tag device 20B. The reader 10B obtains detection information on the basis of only the detection signal component having the larger intensity among the first detection signal component and the second detection signal component of the received detection signal. Thus, the reader 10B obtains the detection information on the basis of only the first detection signal component among the first detection signal component and the second detection signal component of the received detection signal.

The reader 10B then ends either the stand-by for reception and the transmission of the signal having a carrier wave having the first frequency or the stand-by for reception and the transmission of the signal having a carrier wave having the second frequency, on the basis of the detected result indicated by the obtained detection information. Since the detected result indicates that the tag device 20B is outside the living body, the reader 10B ends the transmission of the second request signal and the stand-by for reception of the second detection signal and the second request signal (Step S2041 in FIG. 9).

For the second sub-period of the period during which the third signal component of the request signal is received, the tag device 20B modulates the request signal received by the antenna 210 using the electrical energy stored in the capacitor, on the basis of the response information and transmits the modulated request signal as a response signal (Step S205 in FIG. 9). In this modification, the tag device 20B transmits a response signal representing response information in accordance with a backscattering scheme.

In this case, the carrier wave of the response signal transmitted by the antenna 210 has the first frequency. In other words, the response signal does not contain a component whose carrier wave has the second frequency.

The reader 10B receives the response signal transmitted by the tag device 20B. In this case, the reader 10B obtains the response information on the basis of the first response signal received by the first reception circuit 151.

A case of the tag device 20B inside the living body will now be described. In this case, the antenna 210 of the tag device 20B is in contact with a fluid inside the living body (saliva, in this modification).

The reader 10B starts the transmission of the first request signal and the second request signal, and starts the stand-by for reception of the first detection signal and the first response signal and the stand-by for reception of the second detection signal and the second response signal (S201 in FIG. 10).

The tag device 20B receives the first request signal and the second request signal. Since the antenna 210 is in contact with the fluid inside the living body, the resonance frequency of the antenna 210 is substantially identical to the second frequency. Thus, the intensity of the second request signal received by the antenna 210 is larger than the intensity of the first request signal received by the antenna 210.

The tag device 20B rectifies the current generated between the first antenna component 211 and the second antenna component 212 at the rectifier and stores a charge in the capacitor for at least a portion of the period during which the first signal component of the request signal is received.

The tag device 20B demodulates the second signal component of the request signal for at least a portion of the period during which the second signal component is received, and obtains the identification information on the basis of the demodulated signal, using the electrical energy stored in the capacitor.

The tag device 20B detects whether the tag device 20B is inside the living body with the sensor 223B. In this case, the tag device 20B detects that the tag device 20B is inside the living body (Step S2022 in FIG. 10).

For the first sub-period of the period during which the third signal component of the request signal is received, the tag device 20B modulates the request signal received by the antenna 210 using the electrical energy stored in the capacitor, on the basis of the detection information and transmits the modulated request signal as a detection signal (Step S203 in FIG. 10). In this modification, the tag device 20B transmits a detection signal representing detection information in accordance with a backscattering scheme.

In this modification, the detection signal transmitted by the antenna 210 includes a first detection signal component having a carrier wave having the first frequency (in other words, first component) and a second detection signal component having a carrier wave having the second frequency (in other words, second component). Since the resonance frequency of the antenna 210 is substantially identical to the second frequency, the intensity of the second detection signal component is larger than the intensity of the first detection signal component.

The reader 10B receives the detection signal transmitted by the tag device 20B. The reader 10B obtains detection information on the basis of only the detection signal component having the larger intensity among the first detection signal component and the second detection signal component of the received detection signal. Thus, the reader 10B obtains the detection information on the basis of only the second detection signal component among the first detection signal component and second detection signal component of the received detection signal.

The reader 10B then ends either the stand-by for reception and the transmission of the signal having a carrier wave having the first frequency or the stand-by for reception and the transmission of the signal having a carrier wave having the second frequency, on the basis of the detected result indicated by the obtained detection information. Since the detected results indicates that the tag device 20B is inside the living body, the reader 10B ends the transmission of the first request signal and the stand-by for reception of the first detection signal and the first response signal (Step S2042 in FIG. 10).

For the second sub-period of the period during which the third signal component of the request signal is received, the tag device 20B modulates the request signal received by the antenna 210 using the electrical energy stored in the capacitor, on the basis of the response information and transmits the modulated request signal as a response signal (Step S205 in FIG. 10). In this modification, the tag device 20B transmits a response signal representing response information in accordance with a backscattering scheme.

In this case, the carrier wave of the response signal transmitted by the antenna 210 has the second frequency. In other words, the response signal does not contain a component whose carrier wave has the first frequency.

The reader 10B receives the response signal transmitted by the tag device 20B. In this case, the reader 10B obtains the response information on the basis of the second response signal received by the second reception circuit 152.

As described above, the wireless communication system 1 according to the first modification to the first embodiment operates in the same manner and achieves the same advantages as those of the wireless communication system 1 according to the first embodiment.

The reader 10B of the wireless communication system 1 according to the first modification to the first embodiment controls the frequency of the carrier wave of the signal transmitted by the reader 10B to the first frequency when the tag device 20B is outside the living body and controls the frequency of the carrier wave of the signal transmitted by the reader 10B to the second frequency when the tag device 20B is inside the living body.

Such control can prevent the transmission of the signal having a carrier wave having the first frequency by the reader 10B while the tag device 20B is being inside the living body. The control can also prevent the transmission of the signal having a carrier wave having the second frequency by the reader 10B while the tag device 20B is being outside the living body. Thus, for example, the electrical power consumption of the reader 10B can be reduced compared to that of the reader 10B for the transmission of both the signal having a carrier wave having the first frequency and the signal having a carrier wave having the second frequency.

When the stand-by for reception of and the transmission of either the signal having a carrier wave having the first frequency or the signal having a carrier wave having the second frequency continue for a threshold time or longer, the reader 10B may resume the stand-by and the transmission of the other one of the signal having a carrier wave having the first frequency and the signal having a carrier wave having the second frequency.

Thus, the reader 10B can communicate with the tag device 20B via the antenna 210 of the tag device 20B regardless of whether the tag device 20B is move to the inside or outside of the living body.

### Second Embodiment

A wireless communication system according to a second embodiment will now be described. The wireless communication system according to the second embodiment differs from the wireless communication system according to the first embodiment in that the tag device is of an active type. The wireless communication system will now be described with focus on the difference. In the second embodiment, components that are identical or very similar to those according to the first embodiment are indicated by the same reference signs.

(Configuration of Reader)

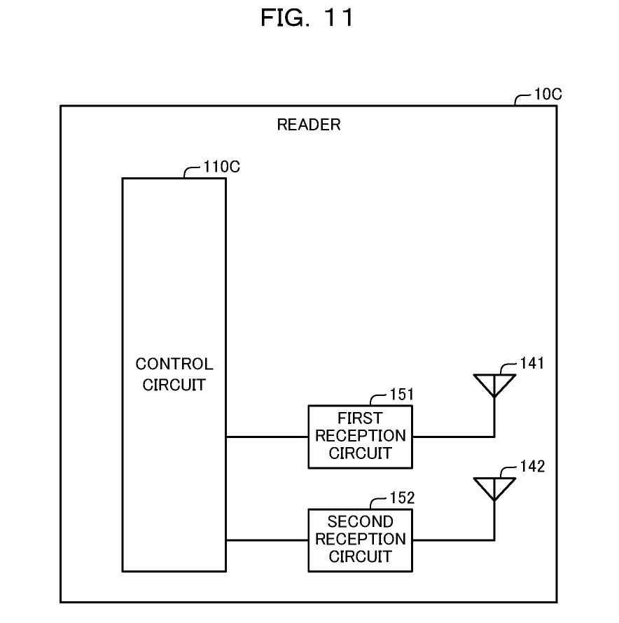

With reference to FIG. 11, a reader 10C according to the second embodiment includes a control circuit 110C, a first reception antenna 141, a second reception antenna 142, a first reception circuit 151, and a second reception circuit 152.

In this embodiment, the first reception circuit 151 and the second reception circuit 152 correspond to receivers. In this embodiment, the control circuit 110C corresponds to a controller.

The first reception circuit 151 receives an broadcast (in other words, advertisement or announcement) signal having a carrier wave having a first frequency (in other words, first broadcast signal) via the first reception antenna 141. In this embodiment, the broadcast signal represents broadcast information. The broadcast information contains at least one of the information stored in the tag device 20C and the information generated by the tag device 20C. In this embodiment, the first frequency is included in a first frequency band among multiple frequency bands collectively referred to as an ISM band. In this embodiment, the first frequency is included in the 5.8 GHz band (from 5.725 to 5.875 GHz, for example).

The second reception circuit 152 receives an broadcast signal having a carrier wave having a second frequency (in other words, second broadcast signal) via the second reception antenna 142. The second frequency is lower than the first frequency. In this embodiment, the second frequency is lower than half the first frequency. In this embodiment, the second frequency is included in a second frequency band, which is lower than the first frequency band, among multiple frequency bands collectively referred to as an ISM band. In this embodiment, the second frequency band is included in the 2.45 GHz band (from 2.4 to 2.5 GHz, for example).

The control circuit 110C controls the first reception circuit 151 to start the stand-by for reception of the broadcast signal. The control circuit 110C also controls the first reception circuit 151 to end the stand-by for reception of the broadcast signal. The control circuit 110C controls the second reception circuit 152 in a similar manner to the first reception circuit 151.

In this embodiment, the control circuit 110C instructs the first reception circuit 151 and the second reception circuit 152 to simultaneously start the stand-by for reception of the broadcast signal.

The control circuit 110C may control the first reception circuit 151 and the second reception circuit 152 so as to alternately enter the stand-by for reception of the broadcast signal by the first reception circuit 151 and the stand-by for reception of the broadcast signal by the second reception circuit 152.

In this embodiment, the control circuit 110C obtains broadcast information on the basis of only the signal having the larger intensity among the first broadcast signal received by the first reception circuit 151 and the second broadcast signal received by the second reception circuit 152.

The control circuit 110C may detect that the tag device 20C has been introduced from the outside to the inside of the living body on the basis of the intensity of the first broadcast signal received by the first reception circuit 151 and the second broadcast signal received by the second reception circuit 152. In such a case, the control circuit 110C may detect that the tag device 20C has been introduced from the outside to the inside of the living body through a variation in the intensity of the first broadcast signal from being larger than the intensity of the second broadcast signal to being smaller than the intensity of the second broadcast signal.

The control circuit 110C may detect that the tag device 20C has been moved from the inside to the outside of the living body on the basis of the intensity of the first broadcast signal received by the first reception circuit 151 and the intensity of the second broadcast signal received by the second reception circuit 152. In such a case, the control circuit 110C may detect that the tag device 20C has been moved from the inside to the outside of the living body through a variation in the intensity of the second broadcast signal from being larger than the intensity of the first broadcast signal to being smaller than the intensity of the first broadcast signal.

(Configuration of Tag Device)

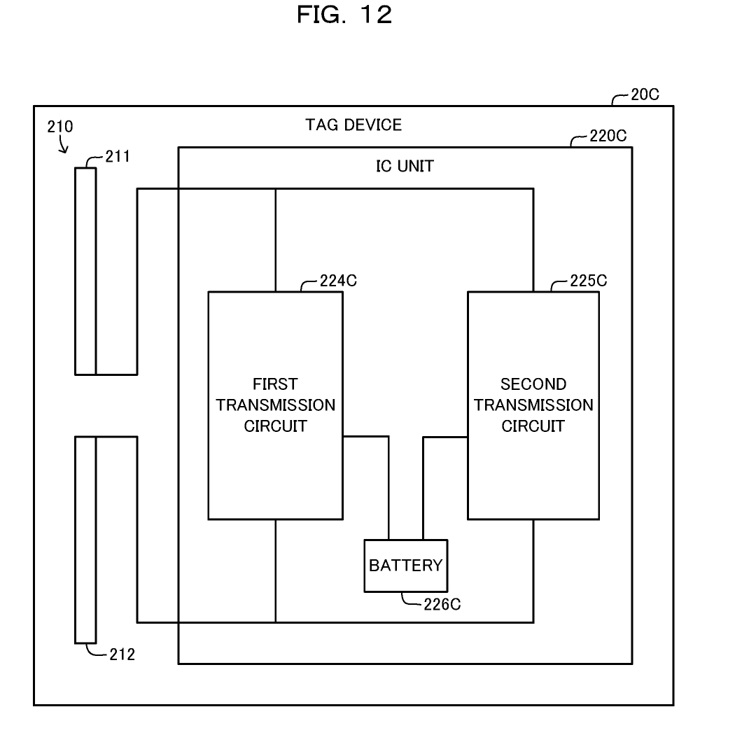

With reference to FIG. 12, the tag device 20C according to the second embodiment includes an antenna 210 and an IC unit 220C. In this embodiment, the IC unit 220C corresponds to a transmitter.

The antenna 210 has a configuration that is the same as that of the antenna 210 according to the first embodiment.

The IC unit 220C is in connection with the first antenna component 211 and the second antenna component 212. The IC unit 220C includes a first transmission circuit 224C, a second transmission circuit 225C, and a battery 226C. In this embodiment, the tag device 20C is of an active type.

The first transmission circuit 224C transmits an broadcast signal having a carrier wave having the first frequency (in other words, first broadcast signal) via the antenna 210, using the electrical energy stored in the battery 226C. The first transmission circuit 224C transmits the first broadcast signal modulated in accordance with a preselected first modulation scheme. The first modulation scheme may be an AM, FM, or PM scheme, for example. Alternatively, the first modulation scheme may be a combination of at least two of the AM, FM, and PM schemes, for example.

The second transmission circuit 225C transmits an broadcast signal having a carrier wave having a second frequency (in other words, second broadcast signal) via the antenna 210, using the electrical energy stored in the battery 226C. The second transmission circuit 225C transmits the second broadcast signal modulated in accordance with a preselected second modulation scheme. The second modulation scheme may be an AM, FM, or PM scheme, for example. Alternatively, the second modulation scheme may be a combination of at least two of the AM, FM, and PM schemes, for example.

In this embodiment, the broadcast information contains information stored in the IC unit 220C in advance. For example, the broadcast information may contain an identifier for identifying the tag device 20C. If the tag device 20C includes a sensor for detecting a physical quantity, the broadcast information may contain information representing the physical quantity detected by the sensor in addition to or instead of the information stored in the IC unit 220C in advance. The physical quantity is temperature, humidity, illuminance, pH, acceleration, angular velocity, pressure, or concentration of a subject, for example. Examples of the subject include a digestive fluid (e.g., saliva, gastric juice, intestinal juice, or pancreatic juice), blood, normal microbial flora, and infectious material (for example, bacteria or viruses).

The tag device 20C may include a first battery in connection with the first transmission circuit 224C and a second battery in connection with the second transmission circuit 225C, in addition to or in place of the battery 226C.

(Operation)

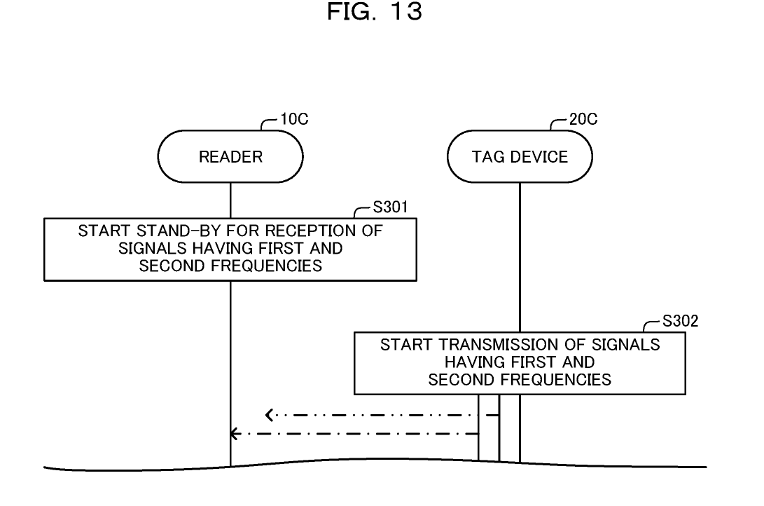

The operation of the wireless communication system 1 will now be explained with reference to FIG. 13.

A case of the tag device 20C outside the living body will now be explained. In this case, the antenna 210 of the tag device 20C is in contact with air.

The reader 10C starts the stand-by for reception of an broadcast signal having a carrier wave having the first frequency (in other words, first broadcast signal) and the stand-by for reception of an broadcast signal having a carrier wave having the second frequency (in other words, second broadcast signal) (Step S301 in FIG. 13).

The tag device 20C starts the transmission of the first broadcast signal and the second broadcast signal (Step S302 in FIG. 13).

The broadcast signal transmitted by the antenna 210 contains a first broadcast signal component having a carrier wave having the first frequency (in other words, first component) and a second broadcast signal component having a carrier wave having the second frequency (in other words, second component). Since the antenna 210 is in contact with air, the resonance frequency of the antenna 210 is substantially identical to the first frequency. Thus, the intensity of the first broadcast signal component transmitted by the antenna 210 is larger than the intensity of the second broadcast signal component transmitted by the antenna 210.

The reader 10C receives the broadcast signal transmitted by the tag device 20C. The reader 10C obtains broadcast information on the basis of only the broadcast signal component having the larger intensity among the first broadcast signal component and the second broadcast signal component of the received broadcast signal. Thus, the reader 10C obtains the broadcast information on the basis of only the first broadcast signal component among the first broadcast signal component and the second broadcast signal component of the received broadcast signal.

A case of the tag device 20C inside the living body will now be described. In this case, the antenna 210 of the tag device 20C is in contact with a fluid inside the living body (saliva, in this embodiment).

Similar to the operation explained above, the reader 10C starts the stand-by for reception of a first broadcast signal component and a second broadcast signal component.

The tag device 20C starts the transmission of the first broadcast signal component and the second broadcast signal component.

The broadcast signal transmitted by the antenna 210 contains a first broadcast signal component having a carrier wave having the first frequency (in other words, first component) and a second broadcast signal component having a carrier wave having the second frequency (in other words, second component). Since the antenna 210 is in contact with the fluid inside the living body, the resonance frequency of the antenna 210 is substantially identical to the second frequency. Thus, the intensity of the second broadcast signal component transmitted by the antenna 210 is larger than the intensity of the first broadcast signal component transmitted by the antenna 210.

The reader 10C receives the broadcast signal transmitted by the tag device 20C. The reader 10C obtains the broadcast information on the basis of only the broadcast signal component having the larger intensity among the first broadcast signal component and the second broadcast signal component of the received broadcast signal. Thus, the reader 10C obtains the broadcast information on the basis of only the second broadcast signal component among the first broadcast signal component and the second broadcast signal component of the received broadcast signal.

As described above, the wireless communication system 1 according to the second embodiment carries out communication between the tag device 20C and the reader 10C via the antenna 210 at the first frequency when the tag device 20C is outside the living body. The wireless communication system 1 also carries out communication between the tag device 20C and the reader 10C via the antenna 210 at the second frequency, which is lower than the first frequency, when the tag device 20C is inside the living body.

Thus, the reader 10C can communicates with the tag device 20C via the antenna 210 of the tag device 20C regardless of whether the tag device 20C is inside or outside the living body.

The reader 10C according to the second embodiment receives a signal having the first frequency and a signal having the second frequency.

Thus, the reader 10C can receive a signal having the first frequency when the tag device 20C is outside the living body. The reader 10C can receive a signal having the second frequency when the tag device 20C is inside the living body. Thus, the reader 10C can communicate with the tag device 20C via the antenna 210 of the tag device 20C regardless of whether the tag device 20C is inside or outside the living body.

The tag device 20C according to the second embodiment transmits the signal having the first frequency and the signal having the second frequency via the antenna 210.

Thus, the reader 10C can receive the signal having the first frequency when the tag device 20C is outside the living body. The reader 10C can receive the signal having the second frequency when the tag device 20C is inside the living body. Thus, the reader 10C can communicate with the tag device 20C via the antenna 210 of the tag device 20C regardless of whether the tag device 20C is inside or outside the living body.

The wireless communication system 1 may use a frequency included in the 2.45 GHz band (from 2.4 to 2.5 GHz, for example) as the first frequency and a frequency included in the 900 MHz band (from 915 to 955 MHz, for example) as the second frequency.

Alternatively, the wireless communication system 1 may use a frequency included in the 5.8 GHz band (from 5.725 to 5.875 GHz, for example) as the first frequency and a frequency included in the 900 MHz band (from 915 to 955 MHz, for example) as the second frequency.

Alternatively, the wireless communication system 1 may use a frequency included in the 60 GHz band (from 57 to 66 GHz, for example) as the first frequency and a frequency included in the 5.8 GHz band (from 5.725 to 5.875 GHz, for example) as the second frequency.

Alternatively, the wireless communication system 1 may use a frequency included in the 24 GHz band (from 24 to 24.25 GHz, for example) as the first frequency and a frequency included in the 5.8 GHz band (from 5.725 to 5.875 GHz, for example) as the second frequency.

The tag device 20C may include a modifier that modifies the resonance frequency of the antenna 210. For example, the modifier may include a first antenna extension, a second antenna extension, a first switching device, and a second switching device.

The first antenna extension is in connection with the first antenna component 211 via the first switching device. The first switching device switches between a state in which the first antenna component 211 and the first antenna extension are connected and a state in which the first antenna component 211 and the first antenna extension are disconnected.

The second antenna extension is in connection with the second antenna component 212 via the second switching device. The second switching device switches between a state in which the second antenna component 212 and the second antenna extension are connected and a state in which the second antenna component 212 and the second antenna extension are disconnected.

This can modify the ratio of the first frequency to the second frequency to a value different from the ratio of the permittivity of air to the permittivity of a fluid inside the living body. Thus, the frequency used for communication between the reader 10C and the tag device 20C can be selected from a wider range.

The reader 10C may end the stand-by for reception of the first broadcast signal component of the broadcast signal if the reader 10C receives the broadcast signal and if the intensity of the first broadcast signal component is smaller than the intensity of the second broadcast signal component of the broadcast signal. The reader 10C may end the stand-by for reception of the second broadcast signal component of the broadcast signal if the reader 10C receives the broadcast signal and if the intensity of the second broadcast signal component is smaller than the intensity of the first broadcast signal component of the broadcast signal.

This can reduce the electrical power consumption at the reader 10C during the stand-by for reception of an broadcast signal by the reader 10C.

In the wireless communication system 1, the tag device 20C may start the transmission of the signal having the first frequency and the signal having the second frequency before the reader 10C starts the stand-by for reception of the signal having the first frequency and the signal having the second frequency.

<First Modification to Second Embodiment>

A wireless communication system according to a first modification to the second embodiment will now be described. The wireless communication system according to the first modification to the second embodiment differs from the wireless communication system according to the second embodiment in that the tag device detects whether it is inside a living body and the frequency to be used for communication is controlled on the basis of the detected result. The wireless communication system will now be described with focus on these differences. In the first modification to the second embodiment, components that are identical or very similar to those according to the second embodiment are indicated by the same reference signs.

(Configuration of Tag Device)

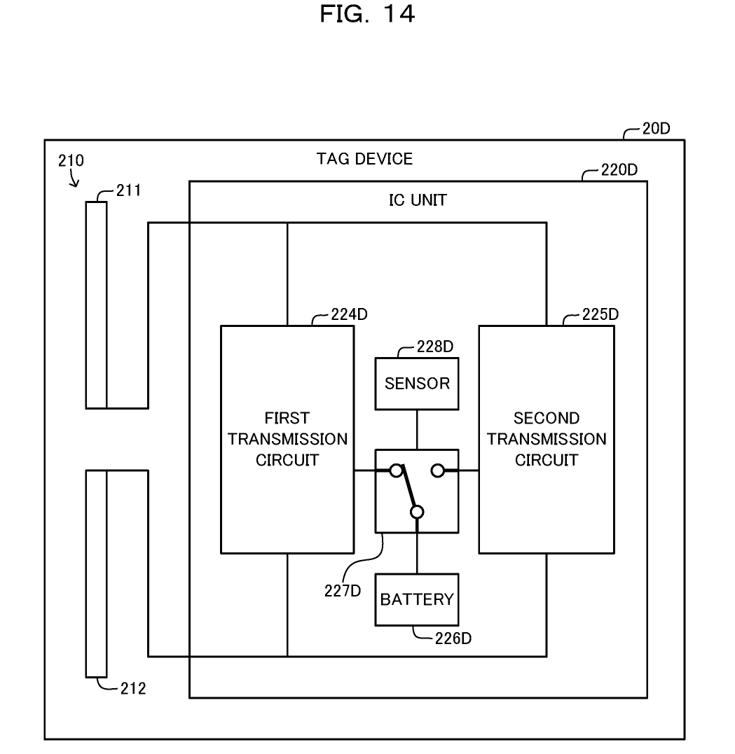

With reference to FIG. 14, a tag device 20D according to the first modification to the second embodiment includes an IC unit 220D in place of the IC unit 220C of the tag device 20C according to the second embodiment. The IC unit 220D includes a first transmission circuit 224D, a second transmission circuit 225D, a battery 226D, a switching device 227D, and a sensor 228D. In this modification, the sensor 228D corresponds to a detector. In this modification, the IC unit 220D corresponds to a transmitter.

The sensor 228D detects whether the tag device 20D is inside a living body. In this modification, the sensor 228D detects temperature. The sensor 228D detects that the tag device 20D is inside the living body when the detected temperature is higher than or equal to a predetermined threshold (307K, for example) and detects that the tag device 20D is outside the living body when the detected temperature is lower than the threshold.

In addition to or instead of detecting temperature, the sensor 228D may detect whether the tag device 20D is inside the living body on the basis of a physical quality other than temperature. The physical quantity may be illuminance, pH, or concentration of a subject, for example. Examples of the subject include a digestive fluid (e.g., saliva, gastric juice, intestinal juice, or pancreatic juice), blood, and normal microbial flora.

In this modification, the sensor 228D operates by the electrical energy stored in the battery 226D.

The switching device 227D switches between a first connection state and a second connection state on the basis of the detected result by the sensor 228D. In the first connection state, the switching device 227D connects the first transmission circuit 224D and the battery 226D and cuts off or disconnects the second transmission circuit 225D and the battery 226D. In the second connection state, the switching device 227D disconnects the first transmission circuit 224D and the battery 226D and connects the second transmission circuit 225D and the battery 226D.

The switching device 227D switches to the first connection state when the detected result by the sensor 228D indicates that the tag device 20D is outside the living body. The switching device 227D switches to the second connection state when the detected result by the sensor 228D indicates that the tag device 20D is inside the living body.

In this modification, the switching device 227D operates by the electrical energy stored in the battery 226D.

When the switching device 227D is in the first connection state, the first transmission circuit 224D transmits an broadcast signal having a carrier wave having a first frequency (in other words, first broadcast signal) via the antenna 210, using the electrical energy stored in the battery 226D. The first transmission circuit 224D transmits the first broadcast signal modulated in accordance with a preselected first modulation scheme. The first modulation scheme may be an AM, FM, or PM scheme, for example. Alternatively, the first modulation scheme may be a combination of at least two of the AM, FM, and PM schemes, for example.

When the switching device 227D is in the second connection state, the second transmission circuit 225D transmits an broadcast signal having a carrier wave having a second frequency (in other words, second broadcast signal) via the antenna 210, using the electrical energy stored in the battery 226D. The second transmission circuit 225D transmits the second broadcast signal modulated in accordance with a preselected second modulation scheme. The second modulation scheme may be an AM, FM, or PM scheme, for example. Alternatively, the second modulation scheme may be a combination of at least two of the AM, FM, and PM schemes, for example.

(Operation)

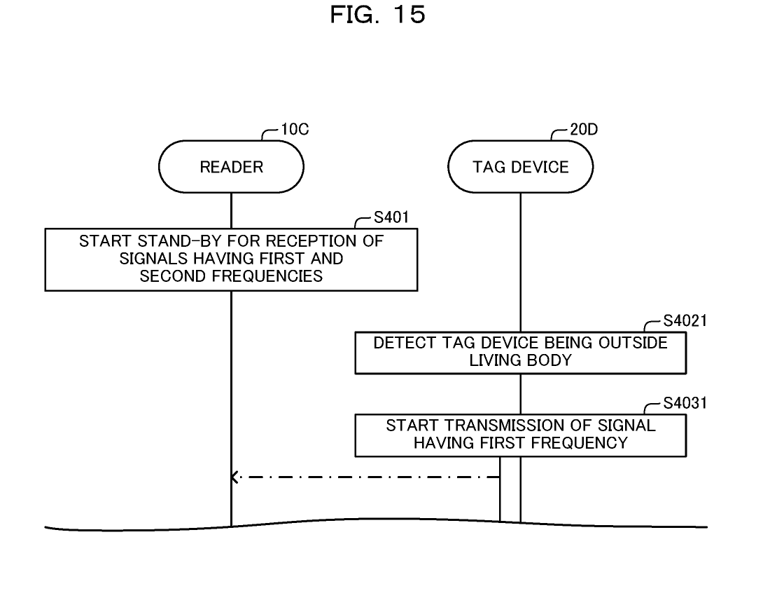

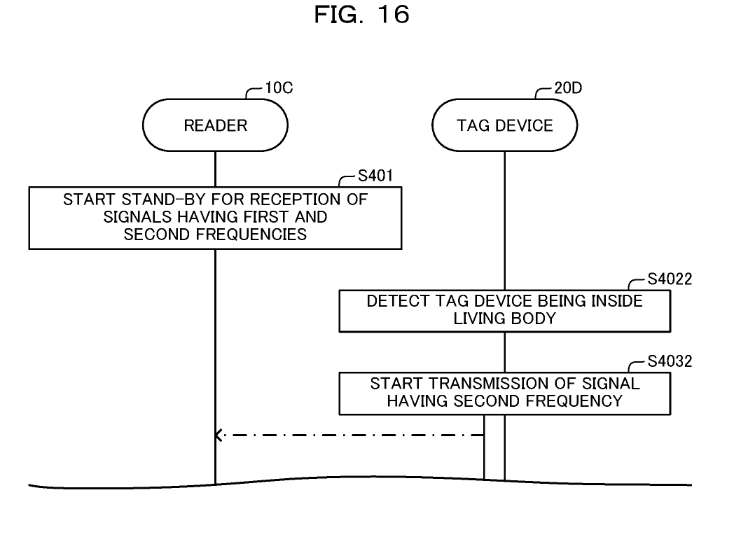

The operation of the wireless communication system 1 according to the first modification to the second embodiment will now be explained with reference to FIGS. 15 and 16.

A case of the tag device 20D outside the living body will now be explained. In this case, the antenna 210 of the tag device 20D is in contact with air.

The reader 10C starts the stand-by for reception of an broadcast signal having a carrier wave having a first frequency (in other words, first broadcast signal) and the stand-by for reception of an broadcast signal having a carrier wave having a second frequency (in other words, second broadcast signal) (Step S401 in FIG. 15).

The tag device 20D detects whether the tag device 20D is inside the living body with the sensor 228D. In this case, the tag device 20D detects that the tag device 20D is not inside the living body (in other words, being outside the living body) (Step S4021 in FIG. 15).

Thus, the switching device 227D switches to the first connection state. This starts the transmission of the first broadcast signal by the tag device 20D (Step S4031 in FIG. 15).

Thus, the carrier wave of the broadcast signal transmitted by the antenna 210 has the first frequency. In other words, the broadcast signal does not contain a component whose carrier wave has the second frequency.

The reader 10C receives the broadcast signal transmitted by the tag device 20D. In this case, the reader 10C obtains broadcast information on the basis of the first broadcast signal received by the first reception circuit 151.

A case of the tag device 20D inside the living body will now be explained. In this case, the antenna 210 of the tag device 20D is in contact with a fluid inside the living body (saliva, in this modification).

The reader 10C starts the stand-by for reception of the first broadcast signal and the stand-by for reception of the second broadcast signal (S401 in FIG. 16).

The tag device 20D detects whether the tag device 20D is inside the living body with the sensor 228D. In this case, the tag device 20D detects that the tag device 20D is inside the living body (Step S4022 in FIG. 16).

Thus, the switching device 227D switches to the second connection state. This starts the transmission of the second broadcast signal by the tag device 20D (Step S4032 in FIG. 16).

Thus, the carrier wave of the broadcast signal transmitted by the antenna 210 has the second frequency. In other words, the broadcast signal does not contain a component whose carrier wave has the first frequency.

The reader 10C receives the broadcast signal transmitted by the tag device 20D. In this case, the reader 10C obtains the broadcast information on the basis of the second broadcast signal received by the second reception circuit 152.

As explained above, the wireless communication system 1 according to the first modification to the second embodiment operates in the same manner and achieves the same advantages as those of the wireless communication system 1 according to the second embodiment.

The tag device 20D of the wireless communication system 1 according to the first modification to the second embodiment controls the frequency of the carrier wave of the signal transmitted by the tag device 20D to the first frequency if the tag device 20D is detected to be outside the living body and controls the frequency of the carrier wave of the signal transmitted by the tag device 20D to the second frequency if the tag device 20D is detected to be inside the living body.

Such control can prevent the transmission of the signal having the first frequency by the tag device 20D while the tag device 20D is being inside the living body. The control can also prevent the transmission of the signal having the second frequency by the tag device 20D while the tag device 20D is being outside the living body. Thus, the electrical power consumption of the tag device 20D can be reduced compared to that of the tag device 20D for transmission of both the signal having the first frequency and the signal having the second frequency.

When the transmission of either the signal having a carrier wave having the first frequency or the signal having a carrier wave having the second frequency continues for a threshold time or longer, the tag device 20D may repeat the detection for whether the tag device 20D is inside the living body and the frequency control on the basis of the detected result.

Thus, the reader 10C can communicate with the tag device 20D via the antenna 210 of the tag device 20D regardless of whether the tag device 20D is move to the inside or outside of the living body.

In the wireless communication system 1, the tag device 20D may start the detection of whether the tag device 20D is inside the living body and the transmission of the signal having the first frequency or the signal having the second frequency before the reader 10C starts the stand-by for reception of the signal having the first frequency and the signal having the second frequency.

### Third Embodiment

A wireless communication system according to a third embodiment will now be described. The wireless communication system according to the third embodiment differs from the wireless communication system according to the first embodiment in that the tag device is of a semi-active type. The wireless communication system will now be described with focus on this difference. In the third embodiment, components that are identical or very similar to those according to the first embodiment are indicated by the same reference signs.

(Configuration of Reader)

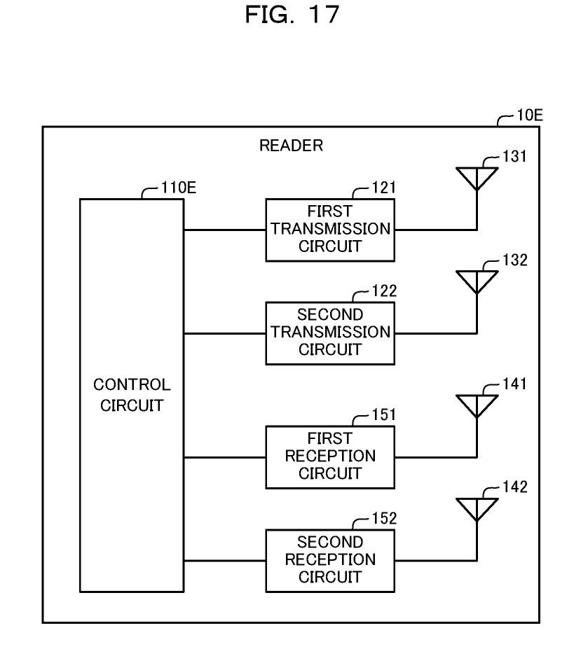

With reference to FIG. 17, a reader 10E according to the third embodiment includes a control circuit 110E, a first transmission circuit 121, a second transmission circuit 122, a first transmission antenna 131, a second transmission antenna 132, a first reception antenna 141, a second reception antenna 142, a first reception circuit 151, and a second reception circuit 152.

In this embodiment, the first transmission circuit 121 and the second transmission circuit 122 correspond to transmitters. In this embodiment, the first reception circuit 151 and the second reception circuit 152 correspond to receivers. In this embodiment, the control circuit 110E corresponds to a controller.

The first transmission circuit 121 transmits a request signal having a carrier wave having a first frequency (in other words, first request signal) via the first transmission antenna 131. In this embodiment, the first frequency is included in a first frequency band among multiple frequency bands collectively referred to as an ISM band. In this embodiment, the first frequency is included in the 5.8 GHz band (from 5.725 to 5.875 GHz, for example). In this embodiment, a request signal requests a tag device 20E, which is described below, to transmit information. In this embodiment, the request signal includes a first signal component and a second signal component, which are continuous along a time axis.

The first signal component is an unmodulated wave (in other words, carrier wave). The second signal component is a modulated wave (in other words, radio wave obtained by modulating a carrier wave). For example, the second signal component represents identification information. The identification information identifies the information requested to be transmitted from the tag device 20E.

In this embodiment, the first signal component and the second signal component have predetermined durations of a first length and a second length, respectively.

The second transmission circuit 122 transmits a request signal having a carrier wave having a second frequency (in other words, second request signal) via the second transmission antenna 132. The second frequency is lower than the first frequency. In this embodiment, the second frequency is lower than half the first frequency. In this embodiment, the second frequency is included in a second frequency band, which is lower than the first frequency band, among multiple frequency bands collectively referred to as an ISM band. In this embodiment, the second frequency band is included in the 2.45 GHz band (from 2.4 to 2.5 GHz, for example).

The first reception circuit 151 receives a response signal having a carrier wave having the first frequency (in other words, first response signal) via the first reception antenna 141. In this embodiment, the response signal represents response information. The response information includes at least one of the information stored in the tag device 20E and the information generated by the tag device 20E, as described below.

The second reception circuit 152 receives a response signal having a carrier wave having the second frequency (in other words, second response signal) via the second reception antenna 142.

The control circuit 110E controls the first transmission circuit 121 to start the transmission of the request signal. The control circuit 110E also controls the first transmission circuit 121 to end the transmission of the request signal. The control circuit 110E controls the second transmission circuit 122 in a similar manner to the first transmission circuit 121.

The control circuit 110E controls the first reception circuit 151 to start the stand-by for reception of the response signal. The control circuit 110E also controls the first reception circuit 151 to end the stand-by for reception of the response signal. The control circuit 110E controls the second reception circuit 152 in a similar manner to the first reception circuit 151.

In this embodiment, the control circuit 110E instructs the first transmission circuit 121 and the second transmission circuit 122 to simultaneously start the transmission of request signals. The control circuit 110E then instructs the first transmission circuit 121 and the second transmission circuit 122 to simultaneously end the transmission of the request signals. The control circuit 110E instructs the first reception circuit 151 and the second reception circuit 152 to simultaneously start the stand-by for reception of the request signals. The control circuit 110E may control the first reception circuit 151 and the second reception circuit 152 to start the stand-by for reception of the request signals substantially simultaneously with the end of the transmission of the request signals.

The control circuit 110E may control the first transmission circuit 121 and the second transmission circuit 122 so as to alternately carry out the transmission of the request signal by the first transmission circuit 121 and the transmission of the request signal by the second transmission circuit 122.

The control circuit 110E may control the first reception circuit 151 and the second reception circuit 152 so as to alternately carry out the stand-by for reception of the response signal by the first reception circuit 151 and the stand-by for reception of the response signal by the second reception circuit 152.

In this embodiment, the control circuit 110E obtains response information on the basis of only the signal having the larger intensity among the first response signal received by the first reception circuit 151 and the second response signal received by the second reception circuit 152.

The control circuit 110E may detect that the tag device 20E has been introduced from the outside to the inside of a living body on the basis of the intensity of the first response signal received by the first reception circuit 151 and the intensity of the second response signal received by the second reception circuit 152. In such a case, the control circuit 110E may detect that the tag device 20E has been introduced from the outside to the inside of the living body through a variation in the intensity of the first response signal from being larger than the intensity of the second response signal to being smaller than the intensity of the second response signal.

The control circuit 110E may detect that the tag device 20E has been moved from the inside to the outside of the living body on the basis of the intensity of the first response signal received by the first reception circuit 151 and the intensity of the second response signal received by the second reception circuit 152. In such a case, the control circuit 110E may detect that the tag device 20E has been moved from the inside to the outside of the living body through a variation in the intensity of the second response signal from being larger than the intensity of the first response signal to being smaller than the intensity of the first response signal.

(Configuration of Tag Device)

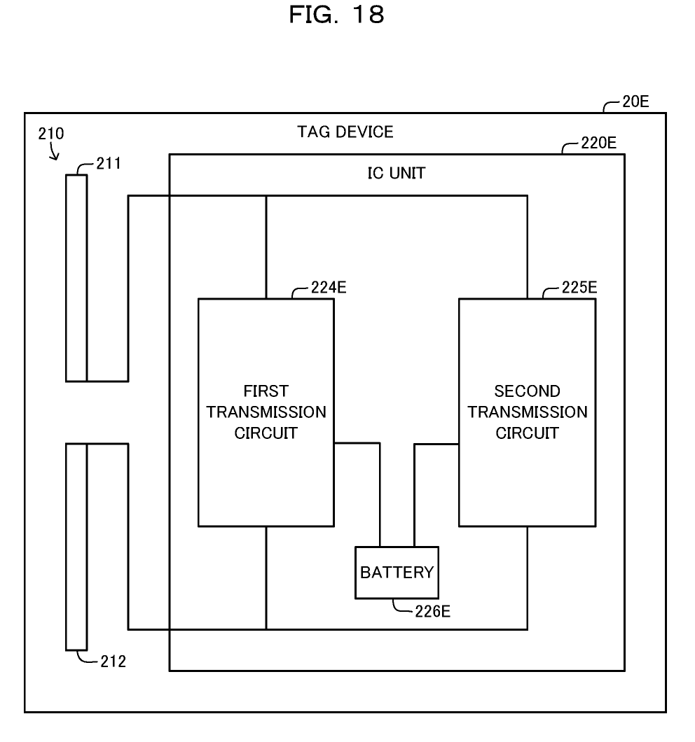

With reference to FIG. 18, the tag device 20E according to the third embodiment includes an antenna 210 and an IC unit 220E. In this embodiment, the IC unit 220E corresponds to a transmitter.

The antenna 210 has a configuration that is the same as that of the antenna 210 according to the first embodiment.

The IC unit 220E is in connection with the first antenna component 211 and the second antenna component 212. The IC unit 220E includes a first transmission circuit 224E, a second transmission circuit 225E, and a battery 226E. In this embodiment, the tag device 20E is of a semi-active type.

In this embodiment, the IC unit 220E includes a rectifier and a capacitor (not illustrated). The current generated between the first antenna component 211 and the second antenna component 212 is rectified at the rectifier and stored in the capacitor for at least a portion of the period during which the first signal component of the request signal is received.

The IC unit 220E demodulates the second signal component for at least a portion of the period during which the second signal component of the request signal is received, using the electrical energy stored in the capacitor. The IC unit 220E obtains identification information on the basis of the demodulated signal.

Alternatively, without using the rectifier and the capacitor (not illustrated), the IC unit 220E may demodulate the second signal component for at least a portion of the period during which the second signal component of the request signal is received, using the electrical energy stored in the battery 226E. In such a case, the request signal does not always include the first signal component.

The first transmission circuit 224E transmits a response signal having a carrier wave having a first frequency (in other words, first response signal) via the antenna 210, using the electrical energy stored in the battery 226E. The first transmission circuit 224E transmits the first response signal modulated in accordance with a preselected first modulation scheme. The first modulation scheme may be an AM, FM, or PM scheme, for example. Alternatively, the first modulation scheme may be a combination of at least two of the AM, FM, and PM schemes, for example.

The second transmission circuit 225E transmits a response signal having a carrier wave having the second frequency (in other words, second response signal) via the antenna 210, using the electrical energy stored in the battery 226E. The second transmission circuit 225E transmits the second response signal modulated in accordance with a preselected second modulation scheme. The second modulation scheme may be an AM, FM, or PM scheme, for example. Alternatively, the second modulation scheme may be a combination of at least two of the AM, FM, and PM schemes, for example.

In this embodiment, the response information contains information stored in the IC unit 220E in advance. For example, the response information may contain an identifier for identifying the tag device 20E. If the tag device 20E includes a sensor for detecting a physical quantity, the response information may contain information representing the physical quantity detected by the sensor in addition to or instead of the information stored in the IC unit 220E in advance. The physical quantity is temperature, humidity, illuminance, pH, acceleration, angular velocity, pressure, or concentration of a subject, for example. Examples of the subject include a digestive fluid (e.g., saliva, gastric juice, intestinal juice, or pancreatic juice), blood, normal microbial flora, and infectious material (for example, bacteria or viruses).

The tag device 20E may include a first battery in connection with the first transmission circuit 224E and a second battery in connection with the second transmission circuit 225E, in addition to or in place of the battery 226E.

(Operation)

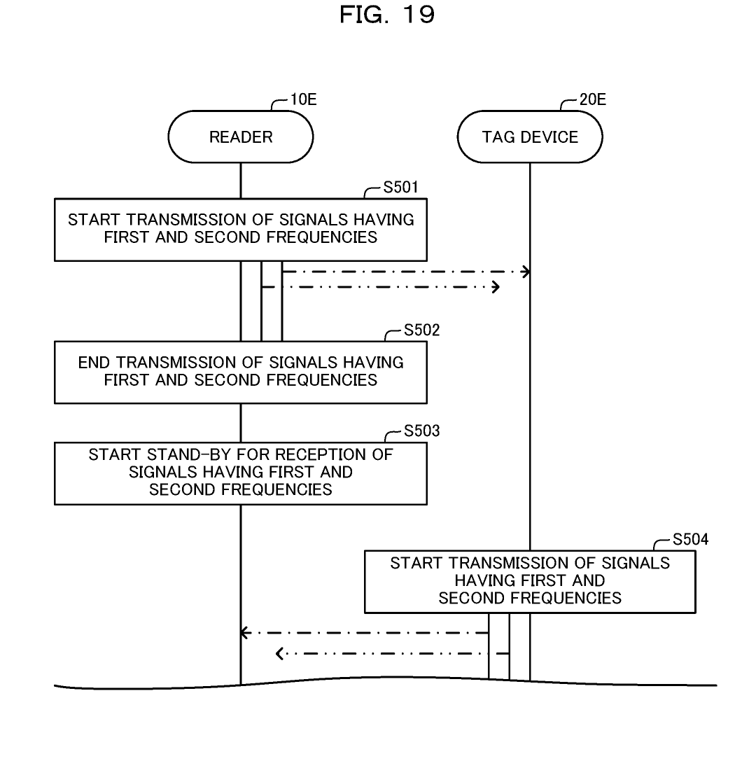

The operation of the wireless communication system 1 according to the third embodiment will now be explained with reference to FIG. 19.

A case of the tag device 20E outside the living body will now be explained. In this case, the antenna 210 of the tag device 20E is in contact with air.

The reader 10E starts the transmission of a request signal having a carrier wave having the first frequency (in other words, first request signal) and the transmission of a request signal having a carrier wave having the second frequency (in other words, second request signal) (Step S501 in FIG. 19).

The tag device 20E receives the first request signal and the second request signal. Since the antenna 210 is in contact with air, the resonance frequency of the antenna 210 is substantially identical to the first frequency. Thus, the intensity of the first request signal received by the antenna 210 is larger than the intensity of the second request signal received by the antenna 210.

The tag device 20E rectifies the current generated between the first antenna component 211 and the second antenna component 212 at the rectifier and stores a charge in the capacitor for at least a portion of the period during which the first signal component of the request signal is received.

The tag device 20E demodulates the second signal component of the request signal for at least a portion of the period during which the second signal component is received, and obtains the identification information on the basis of the demodulated signal, using the electrical energy stored in the capacitor.

The reader 10E ends the transmission of the first request signal and the transmission of the second request signal (Step S502 in FIG. 19).

The reader 10E then starts the stand-by for reception of a response signal having a carrier wave having the first frequency (in other words, first response signal) and the stand-by for reception of a response signal having a carrier wave having the second frequency (in other words, second response signal) (Step S503 in FIG. 19).

The tag device 20E starts the transmission of the first response signal and the transmission of the second response signal (Step S504 in FIG. 19).

The response signal transmitted by the antenna 210 includes a first response signal component having a carrier wave having the first frequency (in other words, first component) and a second response signal component having a carrier wave having the second frequency (in other words, second component). Since the antenna 210 is in contact with air, the resonance frequency of the antenna 210 is substantially identical to the first frequency. Thus, the intensity of the first response signal component transmitted by the antenna 210 is larger than the intensity of the second response signal component transmitted by the antenna 210.

The reader 10E receives the response signal transmitted by the tag device 20E. The reader 10E obtains the response information on the basis of only the response signal component having the larger intensity among the first response signal component and the second response signal component of the received response signal. Thus, the reader 10E obtains the response information on the basis of only the first response signal component among the first response signal component and the second response signal component of the received response signal.

A case of the tag device 20E inside the living body will now be described. In this case, the antenna 210 of the tag device 20E is in contact with a fluid inside the living body (saliva, in this embodiment).

Similar to the operation explained above, the reader 10E starts the transmission of the first request signal and the transmission of the second request signal.

The tag device 20E receives the first request signal and the second request signal. Since the antenna 210 is in contact with the fluid inside the living body, the resonance frequency of the antenna 210 is substantially identical to the second frequency. Thus, the intensity of the second request signal received by the antenna 210 is larger than the intensity of the first request signal received by the antenna 210.

The tag device 20E rectifies the current generated between the first antenna component 211 and the second antenna component 212 at the rectifier and stores a charge in the capacitor for at least a portion of the period during which the first signal component of the request signal is received.

The tag device 20E demodulates the second signal component of the request signal for at least a portion of the period during which the second signal component is received, and obtains the identification information on the basis of the demodulated signal, using the electrical energy stored in the capacitor.

The reader 10E ends the transmission of the first request signal and the transmission of the second request signal. The reader 10E then starts the stand-by for reception of the first response signal and the stand-by for reception of the second response signal.

The tag device 20E starts the transmission of the first response signal and the transmission of the second response signal.

The response signal transmitted by the antenna 210 includes a first response signal component having a carrier wave having the first frequency (in other words, first component) and the second response signal component having a carrier wave having the second frequency (in other words, second component). Since the antenna 210 is in contact with the fluid inside the living body, the resonance frequency of the antenna 210 is substantially identical to the second frequency. Thus, the intensity of the second response signal component transmitted by the antenna 210 is larger than the intensity of the first response signal component transmitted by the antenna 210.

The reader 10E receives the response signal transmitted by the tag device 20E. The reader 10E obtains the response information on the basis of only the response signal component having the larger intensity among the first response signal component and the second response signal component of the received response signal. Thus, the reader 10E obtains the response information on the basis of only the second response signal component among the first response signal component and the second response signal component of the received response signal.

As described above, the wireless communication system 1 according to the third embodiment carries out communication between the tag device 20E and the reader 10E via the antenna 210 at the first frequency when the tag device 20E is outside the living body. The wireless communication system 1 carries out communication between the tag device 20E and the reader 10E via the antenna 210 at the second frequency, which is lower than the first frequency, when the tag device 20E is inside the living body.

Thus, the reader 10E can communicate with the tag device 20E via the antenna 210 of the tag device 20E regardless of whether the tag device 20E is inside or outside the living body.

The reader 10E according to the third embodiment receives a signal having the first frequency and a signal having the second frequency.

Thus, the reader 10E can receive the signal having the first frequency when the tag device 20E is outside the living body. The reader 10E can receive the signal having the second frequency when the tag device 20E is inside the living body. Thus, the reader 10E can communicate with the tag device 20E via the antenna 210 of the tag device 20E regardless of whether the tag device 20E is inside or outside the living body.

The tag device 20E according to the third embodiment transmits the signal having the first frequency via the antenna 210 and transmits the signal having the second frequency via the antenna 210.

Thus, the reader 10E can receive the signal having the first frequency when the tag device 20E is outside the living body. The reader 10E can receive the signal having the second frequency when the tag device 20E is inside the living body. Thus, the reader 10E can communicate with the tag device 20E via the antenna 210 of the tag device 20E regardless of whether the tag device 20E is inside or outside the living body.

The reader 10E according to the third embodiment transmits the signal having the first frequency and the signal having the second frequency.

Thus, the tag device 20E can receive the signal having the first frequency when the tag device 20E is outside the living body. The tag device 20E can receive the signal having the second frequency when the tag device 20E is inside the living body. Thus, the tag device 20E can communicate with the reader 10E via the antenna 210 of the tag device 20E regardless of whether the tag device 20E is inside or outside the living body.

The wireless communication system 1 may use a frequency included in the 2.45 GHz band (from 2.4 to 2.5 GHz, for example) as the first frequency and a frequency included in the 900 MHz band (from 915 to 955 MHz, for example) as the second frequency.

Alternatively, the wireless communication system 1 may use a frequency included in the 5.8 GHz band (from 5.725 to 5.875 GHz, for example) as the first frequency and a frequency included in the 900 MHz band (from 915 to 955 MHz, for example) as the second frequency.

Alternatively, the wireless communication system 1 may use a frequency included in the 60 GHz band (from 57 to 66 GHz, for example) as the first frequency and a frequency included in the 5.8 GHz band (from 5.725 to 5.875 GHz, for example) as the second frequency.

Alternatively, the wireless communication system 1 may use a frequency included in the 24 GHz band (from 24 to 24.25 GHz, for example) as the first frequency and a frequency included in the 5.8 GHz band (from 5.725 to 5.875 GHz, for example) as the second frequency.

The tag device 20E may include a modifier that modifies the resonance frequency of the antenna 210. For example, the modifier may include a first antenna extension, a second antenna extension, a first switching device, and a second switching device.

The first antenna extension is in connection with the first antenna component 211 via the first switching device. The first switching device switches between a state in which the first antenna component 211 and the first antenna extension are connected and a state in which the first antenna component 211 and the first antenna extension are disconnected.

The second antenna extension is in connection with the second antenna component 212 via the second switching device. The second switching device switches between a state in which the second antenna component 212 and the second antenna extension are connected and a state in which the second antenna component 212 and the second antenna extension are disconnected.

This can modify the ratio of the first frequency to the second frequency to a value different from the ratio of the permittivity of air to the permittivity of a fluid inside the living body. Thus, the frequency used for communication between the reader 10E and the tag device 20E can be selected from a wider range.

The reader 10E may end the stand-by for reception of the first response signal component of the response signal if the reader 10E receives the response signal and if the intensity of the first response signal component is smaller than the intensity of the second response signal component of the response signal. The reader 10E may end the stand-by for reception of the second response signal component of the response signal if the reader 10E receives the response signal and if the intensity of the second response signal component is smaller than the intensity of the first response signal component of the response signal.

This can reduce the electrical power consumption at the reader 10E during the stand-by for reception of a response signal by the reader 10E.

In the wireless communication system 1, the tag device 20E may start the transmission of the signal having the first frequency and the signal having the second frequency before the reader 10E starts the stand-by for reception of the signal having the first frequency and the signal having the second frequency.

<First Modification to Third Embodiment>

A wireless communication system according to a first modification to the third embodiment will now be described. The wireless communication system according to the first modification to the third embodiment differs from the wireless communication system according to the third embodiment in that the tag device detects whether it is inside a living body and the frequency to be used for communication is controlled on the basis of the detected result. The wireless communication system will now be described with focus on these differences. In the first modification to the third embodiment, components that are identical or very similar to those according to the third embodiment are indicated by the same reference signs.

(Configuration of Tag Device)

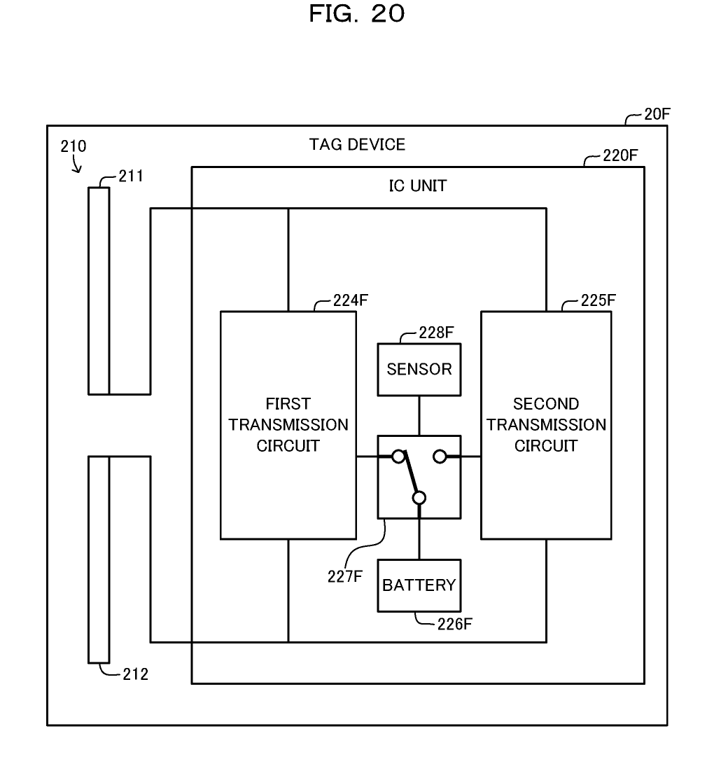

With reference to FIG. 20, a tag device 20F according to the first modification to the third embodiment includes an IC unit 220F in place of the IC unit 220E of the tag device 20E according to the third embodiment. The IC unit 220F includes a first transmission circuit 224F, a second transmission circuit 225F, a battery 226F, a switching device 227F, and a sensor 228F. In this modification, the sensor 228F corresponds to a detector. In this modification, the IC unit 220F corresponds to a transmitter.

In this modification, the IC unit 220F includes a rectifier and a capacitor (not illustrated). The current generated between the first antenna component 211 and the second antenna component 212 is rectified at the rectifier and stored in the capacitor for at least a portion of the period during which the first signal component of the request signal is received.

The IC unit 220F demodulates the second signal component for at least a portion of the period during which the second signal component of the request signal is received, using the electrical energy stored in the capacitor. The IC unit 220F obtains identification information on the basis of the demodulated signal.

Alternatively, without using the rectifier and the capacitor (not illustrated), the IC unit 220F may demodulate the second signal component for at least a portion of the period during which the second signal component of the request signal is received, using the electrical energy stored in the battery 226F. In such a case, the request signal does not always include the first signal component.

The sensor 228F detects whether the tag device 20F is inside the living body. In this modification, the sensor 228F detects temperature. The sensor 228F detects that the tag device 20F is inside the living body when the detected temperature is higher than or equal to a predetermined threshold (307K, for example) and detects that the tag device 20F is outside the living body when the detected temperature is lower than the threshold.

In addition to or instead of detecting temperature, the sensor 228F may detect whether the tag device 20F is inside the living body on the basis of a physical quality other than temperature. The physical quantity may be illuminance, pH, or concentration of a subject, for example. Examples of the subject include a digestive fluid (e.g., saliva, gastric juice, intestinal juice, or pancreatic juice), blood, and normal microbial flora.

In this modification, the sensor 228F operates by the electrical energy stored in the capacitor. Alternatively, the sensor 228F may operate by the electrical energy stored in the battery 226F.

The switching device 227F switches between a first connection state and a second connection state on the basis of the detected result by the sensor 228F. In the first connection state, the switching device 227F connects the first transmission circuit 224F and the battery 226F and cuts off or disconnects the second transmission circuit 225F and the battery 226F. In the second connection state, the switching device 227F disconnects the first transmission circuit 224F and the battery 226F and connects the second transmission circuit 225F and the battery 226F.

The switching device 227F switches to the first connection state when the detected result by the sensor 228F indicates that the tag device 20F is outside the living body. The switching device 227F switches to the second connection state when the detected result by the sensor 228F indicates that the tag device 20F is inside the living body.

In this modification, the switching device 227F operates by the electrical energy stored in the capacitor. Alternatively, the switching device 227F may operate by the electrical energy stored in the battery 226F.

The first transmission circuit 224F transmits a response signal having a carrier wave having a first frequency (in other words, first response signal) via the antenna 210 when the switching device 227F is in the first connection state, using the electrical energy stored in the battery 226F. The first transmission circuit 224F transmits the first response signal modulated in accordance with a preselected first modulation scheme. The first modulation scheme may be an AM, FM, or PM scheme, for example. Alternatively, the first modulation scheme may be a combination of at least two of the AM, FM, and PM schemes, for example.

The second transmission circuit 225F transmits a response signal having a carrier wave having a second frequency (in other words, second response signal) via the antenna 210 when the switching device 227F is in the second connection state, using the electrical energy stored in the battery 226F. The second transmission circuit 225F transmits the second response signal modulated in accordance with a preselected second modulation scheme. The second modulation scheme may be an AM, FM, or PM scheme, for example. Alternatively, the second modulation scheme may be a combination of at least two of the AM, FM, and PM schemes, for example.

(Operation)

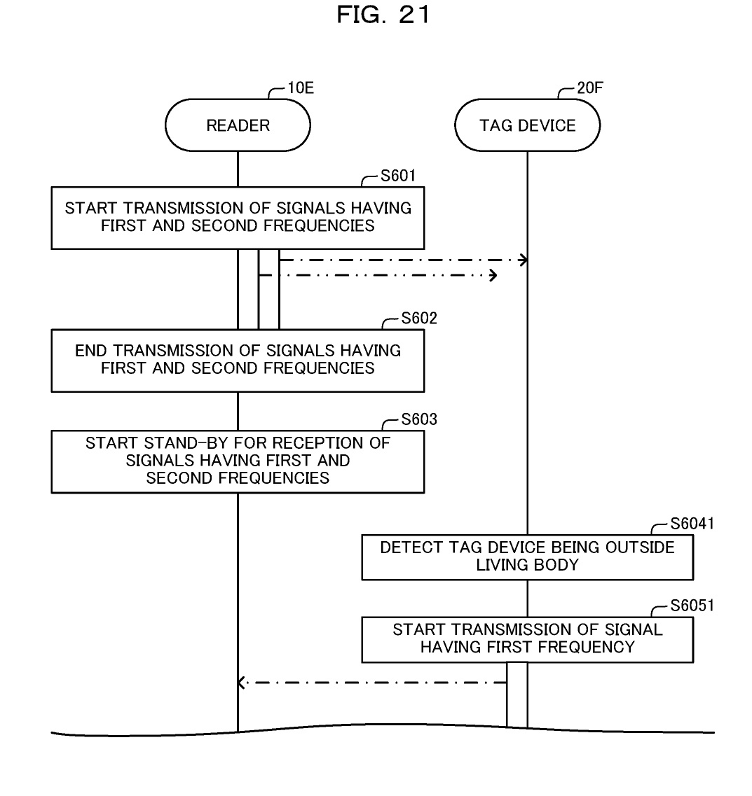

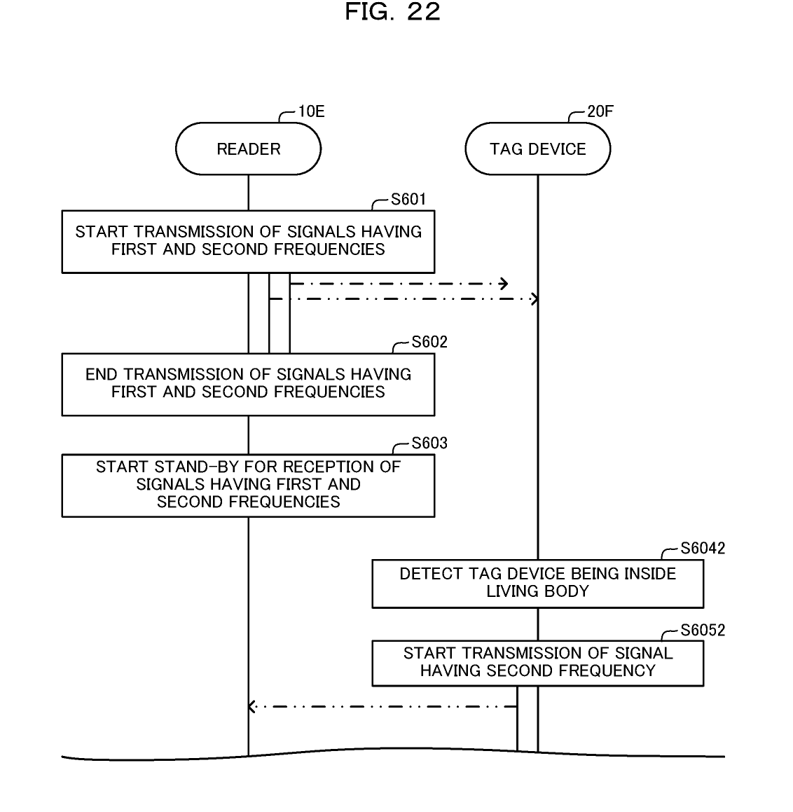

The operation of the wireless communication system 1 according to the first modification to the third embodiment will now be explained with reference to FIGS. 21 and 22.

A case of the tag device 20F outside the living body will now be explained. In this case, the antenna 210 of the tag device 20F is in contact with air.

The reader 10E starts the transmission of a request signal having a carrier wave having the first frequency (in other words, first request signal) and the transmission of a request signal having a carrier wave having the second frequency (in other words, second request signal) (Step S601 in FIG. 21).

The tag device 20F receives the first request signal and the second request signal. Since the antenna 210 is in contact with air, the resonance frequency of the antenna 210 is substantially identical to the first frequency. Thus, the intensity of the first request signal received by the antenna 210 is larger than the intensity of the second request signal received by the antenna 210.

The tag device 20F rectifies the current generated between the first antenna component 211 and the second antenna component 212 at the rectifier and stores a charge in the capacitor for at least a portion of the period during which the first signal component of the request signal is received.

The tag device 20F demodulates the second signal component of the request signal for at least a portion of the period during which the second signal component is received, and obtains the identification information on the basis of the demodulated signal, using the electrical energy stored in the capacitor.

The reader 10E ends the transmission of the first request signal and the transmission of the second request signal (Step S602 in FIG. 21).

The reader 10E then starts the stand-by for reception of a response signal having a carrier wave having the first frequency (in other words, first response signal) and the stand-by for reception of a response signal having a carrier wave having the second frequency (in other words, second response signal) (Step S603 in FIG. 21).

The tag device 20F detects whether the tag device 20F is inside the living body with the sensor 228F. In this case, the tag device 20F detects that the tag device 20F is not inside the living body (in other words, outside the living body) (Step S6041 in FIG. 21).

Thus, the switching device 227F switches to the first connection state. This starts the transmission of the first response signal by the tag device 20F (Step S6051 in FIG. 21).

Thus, the carrier wave of the response signal transmitted by the antenna 210 has the first frequency. In other words, the response signal does not contain a component whose carrier wave has the second frequency.

The reader 10E receives the response signal transmitted by the tag device 20F. In this case, the reader 10E obtains the response information on the basis of the first response signal received by the first reception circuit 151.

A case of the tag device 20F inside the living body will now be described. In this case, the antenna 210 of the tag device 20F is in contact with a fluid inside the living body (saliva, in this embodiment).

Similar to the operation explained above, the reader 10E starts the transmission of the first request signal and the transmission of the second request signal (Step S601 in FIG. 22).

The tag device 20F receives the first request signal and the second request signal. Since the antenna 210 is in contact with the fluid inside the living body, the resonance frequency of the antenna 210 is substantially identical to the second frequency. Thus, the intensity of the second request signal received by the antenna 210 is larger than the intensity of the first request signal received by the antenna 210.

The tag device 20F rectifies the current generated between the first antenna component 211 and the second antenna component 212 at the rectifier and stores a charge in the capacitor for at least a portion of the period during which the first signal component of the request signal is received.

The tag device 20F demodulates the second signal component of the request signal for at least a portion of the period during which the second signal component is received, and obtains the identification information on the basis of the demodulated signal, using the electrical energy stored in the capacitor.

The reader 10E ends the transmission of the first request signal and the transmission of the second request signal (Step S602 in FIG. 22). The reader 10E then starts the stand-by for reception of the first response signal and the stand-by for reception of the second response signal (Step S603 in FIG. 22).

The tag device 20F detects whether the tag device 20F is inside the living body with the sensor 228F. In this case, the tag device 20F detects that the tag device 20F is inside the living body (Step S6042 in FIG. 22).

Thus, the switching device 227F switches to the second connection state. This starts the transmission of the second response signal by the tag device 20F (Step S6052 in FIG. 22).

Thus, the carrier wave of the response signal transmitted by the antenna 210 has the second frequency. In other words, the response signal does not contain a component whose carrier wave has the first frequency.

The reader 10E receives the response signal transmitted by the tag device 20F. In this case, the reader 10E obtains the response information on the basis of the second response signal received by the second reception circuit 152.

As described above, the wireless communication system 1 according to the first modification to the third embodiment operates in the same manner and achieves the same advantages as those of the wireless communication system 1 according to the third embodiment.

The tag device 20F of the wireless communication system 1 according to the first modification to the third embodiment controls the frequency of the carrier wave of the signal transmitted by the tag device 20F to the first frequency if the tag device 20F is detected to be outside the living body and controls the frequency of the carrier wave of the signal transmitted by the tag device 20F to the second frequency if the tag device 20F is detected to be inside the living body.

Such control can prevent the transmission of the signal having the first frequency by the tag device 20F while the tag device 20F is being inside the living body. The control can also prevent the transmission of the signal having the second frequency by the tag device 20F while the tag device 20F is being outside the living body. Thus, the electrical power consumption of the tag device 20F can be reduced compared to that of the tag device 20F for transmission of both the signal having the first frequency and the signal having the second frequency.

When the transmission of either the signal having a carrier wave having the first frequency or the signal having a carrier wave having the second frequency continues for a threshold time or longer, the tag device 20F may repeat the detection for whether the tag device 20F is inside the living body and the frequency control on the basis of the detected result.

Thus, the reader 10E can communicate with the tag device 20F via the antenna 210 of the tag device 20F regardless of whether the tag device 20F is moved to the inside or outside of the living body.

In the wireless communication system 1, the tag device 20F may start the detection of whether the tag device 20F is inside the living body and the transmission of the signal having the first frequency or the signal having the second frequency before the reader 10E starts the stand-by for reception of the signal having the first frequency and the signal having the second frequency.

According to the above-described technologies, it is possible to carry out communication regardless of whether a wireless device is inside or outside a living body.

All examples and conditional language provided herein are intended for pedagogical purposes to aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiment(s) of the present invention have been described in detail, it should be understood that the various changes, substitutions, and alterations could be made hereto without departing from the spirit and scope of the invention.

What is claimed is:

1. A wireless communication system comprising:
a first wireless device configured to be introduced inside a living body, the first wireless device comprising an antenna; and
a second wireless device configured to communicate with the first wireless device via the antenna, wherein,
communication between the first wireless device and the second wireless device via the antenna is carried out at a first frequency when the first wireless device is outside the living body, and
communication between the first wireless device and the second wireless device via the antenna is carried out at a second frequency lower than the first frequency when the first wireless device is inside the living body.

2. The wireless communication system according to claim 1, wherein the second wireless device comprises a receiver configured to receive a signal having the first frequency and a signal having the second frequency.

3. The wireless communication system according to claim 1, wherein
the first wireless device comprises:
a detector configured to detect whether the first wireless device is inside the living body;
a transmitter configured to transmit a signal via the antenna; and
a controller configure to control the frequency of the signal to the first frequency when the first wireless device is detected to be outside the living body, and control the frequency of the signal to the second frequency when the first wireless device is detected to be inside the living body.

4. The wireless communication system according to claim 1, wherein the first wireless device comprises a transmitter configure to transmit a signal having the first frequency via the antenna and a signal having the second frequency via the antenna.

5. The wireless communication system according to claim 1, wherein the second wireless device comprises:
a transmitter configure to transmit a signal; and
a controller configure to control the frequency of the signal to the first frequency when the first wireless device is outside the living body, and control the frequency of the signal to the second frequency when the first wireless device is inside the living body.

6. The wireless communication system according to claim 5, wherein,
the first wireless device comprises:
a detector configured to detect whether the first wireless device is inside the living body; and
a notifier configured to notify the second wireless device of a result of the detection, and
the controller controls the frequency on the basis of the notified result of the detection.

7. The wireless communication system according to claim 1, wherein the second wireless device comprises a transmitter configured to transmit a signal having the first frequency and a signal having the second frequency.

8. The wireless communication system according to claim 7, wherein the first wireless device comprises a transmitter configured to modulate the signal transmitted by the second wireless device and transmit the modulated signal.

9. The wireless communication system according to claim 1, wherein the first wireless device comprises a modifier configured to modify a resonance frequency of the antenna.

10. A method for wireless communication in a wireless communication system comprising a first wireless device including an antenna and configured to be introduced inside a living body and a second wireless device configured to communicate with the first wireless device via the antenna, the method comprising:
carrying out communication between the first wireless device and the second wireless device via the antenna at a first frequency when the first wireless device is outside the living body; and
carrying out communication between the first wireless device and the second wireless device via the antenna at a second frequency lower than the first frequency when the first wireless device is inside the living body.

11. A wireless device configured to be introduced inside a living body comprising:
an antenna;
a detector configured to detect whether the wireless device is inside the living body;
a transmitter configured to transmit a signal via the antenna; and
a controller configured to control the frequency of the signal to a first frequency when the first wireless device is detected to be outside the living body, and controls the frequency of the signal to a second frequency lower than the first frequency when the first wireless device is detected to be inside the living body.

12. A wireless device functioning as a second wireless device configured to communicate with a first wireless device via an antenna of the first wireless device, the first wireless device being configured to be introduced inside a living body, the second wireless device comprising:
a transmitter configured to transmit a signal; and
a controller configured to control the frequency of the signal to a first frequency when the first wireless device is outside the living body, and control the frequency of the signal to a second frequency that is lower than the first frequency when the first wireless device is inside the living body.
